SEQUENCE LISTING (8 sequences)

SEQ ID NO: 1 - DNA, 22 bp, Homo sapiens
  source 1..22: mol_type = other DNA
tgagaactgaattccatgggtt

SEQ ID NO: 2 - DNA, 22 bp, Homo sapiens
  source 1..22: mol_type = other DNA
acagtagtctgcacattggtta

SEQ ID NO: 3 - DNA, 20 bp, Homo sapiens
  source 1..20: mol_type = other DNA
ggatccgagtcacggcacca

SEQ ID NO: 4 - DNA, 23 bp, Homo sapiens
  source 1..23: mol_type = other DNA
taccctgtagaaccgaatttgtg

SEQ ID NO: 5 - DNA, 23 bp, Homo sapiens
  source 1..23: mol_type = other DNA
tagcaccatttgaaatcagtgtt

SEQ ID NO: 6 - DNA, 21 bp, Homo sapiens
  source 1..21: mol_type = other DNA
ctagactgaagctccttgagg

SEQ ID NO: 7 - DNA, 21 bp, Homo sapiens
  source 1..21: mol_type = other DNA
tcgaggagctcacagtctagt

SEQ ID NO: 8 - DNA, 23 bp, Homo sapiens
  source 1..23: mol_type = other DNA
cccagtgttcagactacctgttc

(19) United States
(12) Patent Application Publication
(10) Pub. No.: US 2025/0382671 A1
(43) Pub. Date: Dec. 18, 2025

(54) METHODS OF DETECTION AND ANALYSIS OF NUCLEIC ACID IN CIRCULATING BODILY FLUIDS

(71) Applicant: BRAIN CHEMISTRY LABS, Jackson, WY (US)

(72) Inventors: Sandra Anne BANACK, Jackson, WY (US); Rachael DUNLOP, Jackson, WY (US); Paul Alan COX, Jackson, WY (US)

(21) Appl. No.: 18/974,425

(22) Filed: Dec. 9, 2024

Related U.S. Application Data
(60) Provisional application No. 63/659,497, filed on Jun. 13, 2024.

(51) Int. Cl. C12Q 1/6883; A61K 31/198

(52) U.S. Cl. CPC C12Q 1/6883; A61K 31/198; C12Q 2600/178

(57) ABSTRACT

Presented herein are methods of identifying a subject who has, or is at risk of developing a motor neuron disease, specifically Amyotrophic Lateral Sclerosis (ALS), and/or Primary Lateral Sclerosis (PLS), that includes determining a presence or amount of two or more micro-RNAs (miRNAs) selected from miR-199a-3p, miR-4454, miR-10b-5p, miR-151a-5p, miR-199a-5p, miR-151a-3p, miR-146a-5p, and/or miR-29b-3p in a subject's circulating blood, without determining a presence or amount of the miRNAs from neural-derived exosomes. Also presented herein are methods of preventing, treating, or delaying the onset of a motor neuron disease, specifically ALS and/or PLS.

### PRIORITY

This application claims priority to Provisional Patent Application Ser. No. 63/659,497, filed on Jun. 13, 2024, entitled: “A microRNA Diagnostic Biomarker for Amyotrophic Lateral Sclerosis,” the disclosure of which is incorporated herein in its entirety.

### SEQUENCE LISTING

This application contains a Sequence Listing which has been submitted electronically in WIPO Standard ST.26 (XML format) and is hereby incorporated by reference in its entirety. Said Sequence Listing copy, created on Dec. 9, 2024, is named “042733-0579307_Sequence_listing_ST26.xml” and is 8,192 bytes in size.

### FIELD OF THE EMBODIMENTS

Certain embodiments relate to methods of detecting the presence of, the absence of, or the amount of one or more miRNAs in bodily fluids, including circulating blood, and determining the presence or absence of one or more neurological disorders. Certain embodiments also relate to specific miRNAs, or subsets thereof, that are predictive and/or diagnostic of a motor neuron disease such as amyotrophic lateral sclerosis (ALS) and primary lateral sclerosis (PLS), as well as methods of monitoring treatments, methods of treatment, and kits for diagnostic purposes.

### INTRODUCTION

The need for biomarkers for all neurodegenerative diseases is well established. See, ALS Strategic Plan 2023, available on nih.gov; Vignaroli, et al., “The Need for Biomarkers in the ALS-FTD Spectrum: A Clinical Point of View on the Role of Proteomics,” Proteomes 11 (1): 1-18 (2023). Biomarkers are essential for reducing diagnostic delays and improving disease outcomes as well as for therapeutic drug development. To improve reproducibility and ultimately to achieve biomarker adoption, every biomarker should undergo robust validation (U.S. Department of Health and Human Services. Biomarker Qualification: Evidentiary Framework Guidance for Industry and Staff 2018, available at fda.gov). The most effective biomarkers are reliable measures of disease (or disease state), applicable across drug development trials, and independent of the drug being tested.

Amyotrophic lateral sclerosis (ALS) is a rare neurological disease with an estimated 30,000 active cases per year in the United States. Incident rates in 2018 were 1.6 per 100,000 USA population and prevalence was 9.1 per 100,000 population (Mehta P, et al., “Prevalence of amyotrophic lateral sclerosis in the United States,” Amyotroph Lateral Scler Frontotemporal Degener, August 30:1-7 (2023); doi: 10.1080/21678421.2023.22458584). The disease typically develops during mid-to-late life, progresses rapidly, and is terminal within two to five years but some patients survive much longer. See also, Shefner J M et al., “A proposal for new diagnostic criteria for ALS,” Clin. Neurophysiol, 113: pp 1975-1978 (2020); Brown R H, et al., “Amyotrophic lateral sclerosis,” N Engl J Med., 377 (2): 162-172 (2017). The clinical presentation of ALS is also variable with distal muscle limb weakness more common than bulbar onset (Verma A., “Clinical Manifestation and Management of Amyotrophic Lateral Sclerosis in Amyotrophic Lateral Sclerosis, In: Toshiyuki A, ed. “Amyotrophic Lateral Sclerosis,” Exon Publication; 1-14.7 (2021). Familial ALS represents approximately 10% of all cases with the remaining 90% of sporadic cases occurring in individuals with no known genetic mutations (Masrori P, et al., “Amyotrophic lateral sclerosis: a clinical review,” Eur J Neurol., 27 (10): 1918-1929 (2020). Care for ALS patients remains supportive and palliative, while a cure, and even meaningfully effective therapy, remains elusive. See, Verma A, above.

An ALS diagnosis typically is made by a clinician after observation of progressive degeneration of upper and lower motor neurons using standardized clinical criteria. However, patients frequently experience diagnostic uncertainty resulting in delayed diagnosis, an increased number of physician consultations, and sometimes unnecessary medical procedures (Richards D, et al., “Time to diagnosis and factors affecting diagnostic delay in amyotrophic lateral sclerosis,” J Neurol Sci., 417:117054 (2020), doi: 10.1016/j.jns.2020.117054.9. Misdiagnosis can be as high as 68%, partly because family physicians and some general neurologists outside of large metropolitan areas do not observe many cases in their lifetimes. A robust ALS diagnostic biomarker would give patients a chance at earlier treatment and novel therapeutic intervention. It could also accelerate the testing of new drug candidates in clinical trials (Kiernan M C, et al., “Improving clinical trial outcomes in amyotrophic lateral sclerosis,” Nature Reviews Neurology, 17 (2): 104-118.10 (2021).

ALS biomarker research, both diagnostic (Gomes B C, et al., “Differential Expression of miRNAs in Amyotrophic Lateral Sclerosis Patients,” Mol Neurobiol., August 2:1-14 (2023)), and prognostic, (Magen I, et al., “Circulating miR-181 is a prognostic biomarker for amyotrophic lateral sclerosis,” Nature neuroscience, 24 (11): 1534-1541 (2021)) is on-going, with recent developments in neuroimaging, electrophysiology, and fluid-based markers. See Ilieva H, Vullaganti M, Kwan J., “Advances in molecular pathology, diagnosis, and treatment of amyotrophic lateral sclerosis,” BMJ, 383: c075037 (2023). Imaging biomarkers can be difficult for ALS patients as symptom-related complications make these procedures uncomfortable (Sturmey E, Malaspina A. “Blood biomarkers in ALS: Challenges, applications and novel frontiers,” Acta Neurologica Scandinavica, 146 (4): 375-388 (2022)). Blood or urine-based biomarkers are generally considered preferable to cerebrospinal fluid markers because they are less invasive. Two candidates, neurofilament and p75 neurotrophin receptor, are being investigated as pharmacological markers in conjunction with ALS clinical trials. See also, Shepheard S R, et al., “The extracellular domain of neurotrophin receptor p75 as a candidate biomarker for amyotrophic lateral sclerosis,” PLOS One, 9: e87398 (2014); Jourdi G, et al., “Soluble p75 neurotrophic receptor as a reliable biomarker in neurodegenerative diseases: what is the evidence?”, Neural Regen Res, 19 (3): 536-541 (2024). RNA biomarkers are also of interest as ALS biomarkers and in combination with neurofilament, may have promise in survival prognostication. See Joilin G, et al., “An Overview of MicroRNAs as Biomarkers of ALS,” Front Neurol, 10:186 (2019); Zhu Y, et al., “Evaluating the causal association between microRNAs and amyotrophic lateral sclerosis,” Neurol Sci., 44 (10): 3567-3575 (2023); Shen D, et al., “Single-Cell RNA Sequencing Analysis of Microglia Dissected the Energy Metabolism and Revealed Potential Biomarkers in Amyotrophic Lateral Sclerosis,” Mol Neurobiol. (2023); and Grima N, et al., “RNA sequencing of peripheral blood in amyotrophic lateral sclerosis reveals distinct molecular subtypes: Considerations for biomarker discovery,” Neuropathol Appl Neurobiol. 49 (6): e12943 (2023). A recent study has demonstrated the potential of HDGFL2, a cryptic neoepitope, as a marker of presymptomatic ALS (Irwin K E, et al., “A fluid biomarker reveals loss of TDP-43 splicing repression in presymptomatic ALS-FTD,” Nat Med., 30 (2): 382-393 (2024); doi: 10.1038/s41591-023-02788-5. Erratum in: Nat Med. 2024 Apr. 5: PMID: 38278991; PMCID: PMC10878965.

Exosomes have been shown to comprise nucleic acids, including messenger RNA (mRNA), microRNA (miRNA), and small interfering RNA (siRNA), which can be transferred from one cell to another. Exosomes that are released into the extracellular matrix and taken up by adjacent cells can potentiality transfer information from one cell to another. Such information can be therapeutic or pathogenic. Detecting and analyzing miRNAs associated with neural-derived exosomes (or neural-enriched extracellular vesicles (NEE), and use of such detection and analysis in diagnosing and early detection of motor neuron diseases, such as ALS, is described in U.S. Patent Application Publication No. 2021/0164051, the disclosure of which is incorporated herein by reference in its entirety.

As described therein, neural-derived exosome fractions, and/or fractions comprising portions or components of exosomes of neural cell or neural tissue origin (individually and collectively referred to interchangeably as “a neural-derived exosome fraction” or “neural-enriched exosome fractions”), can be detected, isolated, enriched, or prepared from a sample, for example, a sample obtained from a subject. Thus, a sample may be obtained from a subject, neural derived exosomes, if detected, are isolated and enriched, and then the exosomes are analyzed for the presence, absence, and/or amount of miRNAs present in the neural-derived exosomes. While these methods are useful in early diagnoses and early detection of motor neuron diseases, such as ALS, the method requires at least the isolation of neural derived exosomes from the sample. The use of neural-derived exosomes also is useful in determining correlations between one or more miRNA, typically 2 or 3, and the presence of indication of a neurodegenerative disorder, such that the presence, absence, and/or amount of a group of miRNAs selected using neural-derived exosomes can be used to diagnose, monitor, and assess treatment efficacy for certain neurodegenerative disorders. The inventors did not heretofore believe that analyzing circulating bodily fluids for the presence, absence, and/or amount of miRNAs without exosome isolation would or could provide an accurate detection or diagnosis of motor neuron diseases.

While PLS and ALS have overlapping symptoms, PLS involves only the upper motor neurons (UMN) while ALS involves both UMN and lower motor neurons. PLS also progresses more slowly than ALS resulting in a longer life expectancy after diagnosis. It would be desirable to develop a blood-based diagnostic ALS and PLS biomarker, or biomarker set that could reliably identify patients who had previously been diagnosed using standardized clinical criteria. If such a diagnostic could be discovered, the inventors hypothesized that patterns and concentrations of certain miRNA within the circulating blood system might be useful with sensitivity and specificity to aid in ALS and/or PLS diagnosis, in ALS and/or PLS monitoring, and in assessing efficacies of therapies for ALS and/or PLS with greater speed and accuracy than previous methods that involved isolating exosomes.

While some of the afore-discussed subject matter is discussed with reference to known literature and publications, the embodiments described herein may include one or more known features, without suffering from drawbacks and/or problems previously encountered. In addition, information presented in this Introduction section is not an admission that the information is prior art to the embodiments described herein, expressly including the preceding paragraph.

### SUMMARY

Presented herein, in certain embodiments, are methods of detection and analysis of miRNAs in circulating bodily fluids without exosome isolation. In some embodiments, such methods can be used for the diagnosis and early detection of a variety of motor neuron diseases and disorders. Also presented herein, in certain embodiments, are methods of determining the relationship between the types and amounts of miRNAs and the absence and/or presence of one or more neurological diseases or disorders, and then using that relationship in a method for the diagnosis and early detection of one or more neurological diseases or disorders by detection and analysis of miRNAs in circulating bodily fluids without exosome isolation.

In some aspects, presented herein is method of identifying a subject who has, is at risk of developing a motor neuron disease, or is being tested to exclude a possible future ALS and/or PLS diagnosis comprising: (a) determining a presence or amount of one or more micro-RNAs (miRNAs) in a sample obtained from the subject without determining the presence or amount in neural derived exosomes, wherein the one or more miRNA are selected from the group consisting of at least miR-199a-3p, miR-4454, miR-10b-5p, miR-151a-5p, miR-199a-5p, miR-151a-3p, miR-146a-5p, miR-29b-3p, and (b) determining if the subject has, or is at risk of developing the motor neuron disease according to the presence or amount of the one or more miRNAs in the sample.

In some aspects, presented herein is a method of preventing or treating a motor neuron disease in a subject who has, or is at risk of developing the motor neuron disease, the method comprising: (a) determining a presence or amount of one or more micro-RNAs (miRNAs) in a sample obtained from the subject without determining the presence or amount in neural derived exosomes, wherein the one or more miRNA are selected from the group consisting of miR-199a-3p, miR-4454, miR-10b-5p, miR-151a-5p, miR-199a-5p, miR-151a-3p, miR-146a-5p, miR-29b-3p; (b) determining if the subject has, or is at risk of developing the motor neuron disease according to the presence or amount of the one or more miRNAs in the sample; and (c) administering a motor neuron disease treatment to the subject when the determining of (b) determines that the subject has, or is at risk of developing the motor neuron disease. In some embodiments, a motor neuron disease treatment comprises administering a therapeutically effective amount of L-serine, and the motor neuron disease is ALS and/or PLS.

In some aspects, presented herein is one or more kits useful in carrying out a method of identifying a subject who has, or is at risk of developing a motor neuron disease that includes one or more multi-well plates, with each well containing one or more miRNA primers selected from the group consisting of miR-199a-3p, miR-4454, miR-10b-5p, miR-151a-5p, miR-199a-5p, miR-151a-3p, miR-146a-5p, miR-29b-3p, and a mechanism to calculate the likelihood of a positive or negative diagnosis, and/or probability of having or not having a neurological disease. In one embodiment, the kit includes two well-plates in which one well plate has each well containing one or more miRNA primers selected from the list above, and the other well plate includes a Quality Control (QC)/spike-in/calibration plate in which one or more wells contain QC primers, no-template controls, and/or spike-in primers designed to monitor the success of the method. In another embodiment, the kit includes one well-plate in which the wells include one or more miRNA primers selected from the list above, QC primers, no-template controls, and/or spike-in primers designed to monitor the success of the method. In a further embodiment, the mechanism to calculate the likelihood of a positive or negative diagnosis, and/or probability of having or not having a neurological disease includes software configured to carry out the calculation using random forest machine learning algorithms and/or logistic regression algorithms developed from machine learning classification of known disease and healthy control plasma samples.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a box-plot representation of variability in gene fold expression [2^{-(ΔΔCt)}] in between amyotrophic lateral sclerosis (ALS, n=119), Parkinson's disease (PD, n=20), primary lateral sclerosis (PLS, n=42), and control samples (n=150) represented by boxplot for eight-miRNAs. Center lines show the medians; box limits indicate the 25th and 75th percentiles; whiskers extend 1.5 times the interquartile range from the 25th and 75th percentiles, outliers are represented by circles. Two extreme values (282 and 275) from control samples were removed from miR-4454 to best illustrate the data. See Table 3 for statistical analyses.

FIG. 2 shows a box-plot comparison of the mean normalized copy number (μL) determined using qPCR and digital PCR (dPCR) for eight miRNAs.

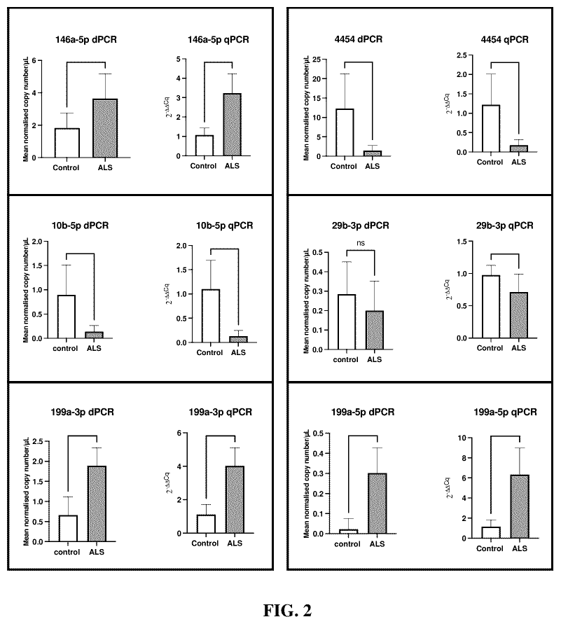

FIG. 3 is a Receiver Operator Characteristics (ROC) curve calculated with a logistic regression model on 269 total observations with 119 ALS and 150 control samples. Area under the curve (AUC)=0.98.

### DETAILED DESCRIPTION

As described herein, the miRNA content of circulating bodily fluids has diagnostic and therapeutic utility. The identification of the specific miRNA useful in detecting certain neurodegenerative disorders, or the risk of developing certain neurodegenerative disorders, such as ALS and/or PLS can be determined by methods disclosed in, for example, U.S. Patent Application Publication No. 2021/0164051, entitled: “Methods of Detection and Analysis of Nucleic Acid in Neural-Derived Exosomes,” the disclosure of which is incorporated by reference herein in its entirety. While the use of exosomes derived from neural cells is useful in the initial identification of miRNA and analyzing the relationships between healthy and un-healthy individuals, using exosomes derived from neural cells in a diagnostic method is complex and cumbersome. The inventors discovered, quite unexpectedly, that detecting the onset or progression or risk of developing a neurodegenerative disorder by determining a presence or amount of one or more micro-RNAs (miRNAs) in a circulating bodily fluid sample (e.g., blood) without isolating and assessing the presence or amount of one or more miRNAs in neural-derived exosomes, was more accurate than determining a presence or amount of one or more miRNAs in exosomes derived from neural cells. Thus, the diagnostic methods described herein are not only simpler and easier than using exosomes derived from neuronal cells, but they are unexpectedly more accurate and predictive.

As discussed above, the techniques described in U.S. Patent Application Publication No. 2021/0164051, can be useful in determining the one or more miRNAs that could be used to identify a subject who has, or is at risk of developing a motor neuron disease. The abundance of, for example, tetraspanins and cell adhesion molecules (CAMs) expressed on or in exosomes derived from neural cells or neural tissues makes it possible to detect, enrich, prepare, and/or isolate neural-derived exosomes. Such neural-derived exosomes may be employed to detect and/or quantify the amount of certain exosome-derived miRNAs. The presence or amount of certain exosome-derived miRNAs, or sets of such miRNAs, provides insight as to whether a subject has, or is at risk of developing certain motor neuron diseases. For example, the presence or amount of certain exosome-derived miRNAs can also be used to provide early diagnosis of a motor neuron disease and/or to identify subjects who are at risk of developing a motor neuron disease. Once one or more miRNAs have been identified using exosomes as being useful in identifying a subject who has or is at risk of developing a motor neuron disease, using the guidelines provided herein, the one or more miRNAs so identified, then can be detected (presence and/or amount) in circulating bodily fluids such as blood without isolating exosomes, and the presence or amount of the miRNAs used to identify such subjects. Such diagnostic methods are far simpler to carry out using circulating bodily fluids, and can be more accurate, when compared to using exosomes derived from neuronal cells.

Amyotrophic Lateral Sclerosis (ALS), a non-limiting example of a motor neuron disease, is the most common form of a motor neuron disease (MND). ALS/MND, or Lou Gehrig's disease, is a progressive motor neuron disease characterized by death of both upper and lower motor neurons and subsequent muscle atrophy. There are a variety of genetic and environmental risk factors that may lead to ALS which is believed to result from gene/environment interactions and this serious illness likely represents a syndrome rather than a single disease. The onset of ALS symptoms often presents a crisis for patients and their families, with time from diagnosis to death having a mean of 2.5 to 3 years, although some patients persist much longer. Loss of functionality due to progressive motor neuron loss leads to ataxia, aphasia, muscle spasticity, muscle fasciculations, and progressive paralysis. Some patients also suffer cognitive deficits.

One of the problems in current ALS therapy is slowness of diagnosis. In its initial presentation, ALS is often misdiagnosed. Few general practitioners feel comfortable in making a possible or probable diagnosis of ALS and so refer patients to neurologists, who in turn typically use progressive deterioration of upper and lower motor neuron function and increasing muscle atrophy as measured through time as indicative of ALS. As a result, diagnosis of ALS typically takes months, and sometimes a year or more, to be made. This precious time lost due to the inability to diagnose presents a significant burden on patients and their families, as well as their physicians, because of the inability to prescribe medication or even to plan for treatment and patient care. At any one time in the United States, there are 25,000 to 30,000 living patients diagnosed with ALS.

In one embodiment, there is provided an ALS and/or PLS diagnostic eight-miRNA fingerprint (miR-199a-3p, miR-4454, miR-10b-5p, miR-151a-5p, miR-199a-5p, miR-151a-3p, miR-146a-5p, miR-29b-3p) obtained from a standard clinical blood draw that may be useful in diagnosing ALS and/or PLS, determining subjects at risk of developing ALS and/or PLS, testing subject to exclude a future ALS and/or PLS diagnosis, and in assessing therapies for ALS and/or PLS. miRNA were identified from an earlier experiment on blood plasma from neural enriched extracellular vesicles in which 101 miRNA were shown by next generation sequencing to be differentially expressed between ALS p and/or PLS patients and healthy controls. See, Banack S A, et al., “An miRNA fingerprint using neural-enriched extracellular vesicles from blood plasma: Towards a biomarker for amyotrophic lateral sclerosis/motor neuron disease,” Open Biology. 10 (6): 200116 (2020). Thirty-four miRNA were selected, due to their magnitude of dysregulation, for validation using qPCR. The eight-miRNA were selected from these 34 miRNA because they consistently and statistically separated ALS and/or PLS patients and control patients following qPCR experiments on three different cohorts of patient and control samples. Banack S A, et al., “miRNA extracted from extracellular vesicles is a robust biomarker of amyotrophic lateral sclerosis,” J. Neurol Sci. 442:120396 (2022). The bilipid membrane of the extracellular vesicles contributed to reproducibility in two ways: 1) protecting miRNA from degradation, and 2) facilitating purification of the sample by immunopurification through transmembrane proteins on the surface of the extracellular vesicles. Dunlop R A, et al., “LICAM immunocapture generates a unique extracellular vesicle population with a reproducible miRNA fingerprint,” RNA biology, 20 (1): 140-148 (2023).

The identified miRNA fingerprint fills an unmet ALS and/or PLS drug development and medical diagnostic need and could facilitate the identification of ALS and/or PLS, or subjects at risk of developing ALS and/or PLS, at its earliest stages, thereby reducing diagnostic uncertainty. The inventors surprisingly discovered that the miRNA fingerprint also was found in circulating blood, without exosome isolation, purification, and analysis, and that the method of diagnosing ALS and/or PLS, identifying subjects at risk of developing ALS and/or PLS, or testing a subject to exclude a future diagnosis of ALS and/or PLS, using this miRNA fingerprint from a standard clinical blood draw was more accurate than prior identification techniques that relied on isolation and analysis of miRNA present within neural-enriched extracellular vesicles. The embodiments described herein present data from a large cohort of patients including ALS and/or PLS, neurological controls with a diagnosis of Parkinson's disease, neurological controls with a diagnosis of primary lateral sclerosis, and healthy controls with no known neurological symptoms that shows that this eight-miRNA fingerprint can be used, along with current diagnostic tools, to aid clinical ALS and/or PLS diagnosis, and identification of subjects at risk of developing ALS and/or PLS.

A “sample” or “samples”, as used interchangeable herein, is often obtained from a suitable subject. A sample can be isolated or obtained directly from a subject or part thereof. In some embodiments, a sample obtained from a subject is a sample derived from the subject. Accordingly, in certain embodiments, a sample obtained from a subject is a sample obtained directly from the subject. In certain embodiments, a sample obtained from a subject is obtained from a third party, for example a third party who obtained or extracted the sample from the subject. In some embodiments, a sample is obtained indirectly from an individual or a medical professional. A sample can be any specimen that is isolated or obtained from a subject or part thereof. Non-limiting examples of samples include fluids or tissues obtained or derived from a subject, including, without limitation, circulating bodily fluids such as blood or a blood product (e.g., serum, plasma, platelets, buffy coats, lymphatic fluid or the like), umbilical cord blood, chorionic villi, amniotic fluid, cerebrospinal fluid (CSF), spinal fluid, lavage fluid (e.g., lung, gastric, peritoneal, ductal, car, arthroscopic), a biopsy sample, celocentesis sample, cells (blood cells, lymphocytes, placental cells, stem cells, bone marrow derived cells, embryo or fetal cells, neurons) or parts thereof (e.g., mitochondrial, nucleus, extracts, lysates, or the like), urine, feces, sputum, saliva, nasal mucous, prostate fluid, lavage, semen, lymphatic fluid, bile, tears, sweat, breast milk, breast fluid, the like or combinations thereof. In an embodiment, a “sample” is blood.

As used herein, the expressions “circulating bodily fluid” or “circulating body fluid” denotes samples, as described above, that circulate throughout the body. Particularly preferred circulating bodily fluids include, but are not limited to, blood, serum, plasma, platelets, buffy coats, lymphatic fluid, urine, semen, spinal fluid, bile, and the like. Blood is especially preferred due to its ease of sampling.

Non-limiting examples of subjects include mammals, humans, non-human primates (e.g., apes, gibbons, chimpanzees, orangutans, monkeys, macaques, and the like), domestic animals (e.g., dogs and cats), farm animals (e.g., horses, cows, goats, sheep, and pigs) and experimental animals (e.g., mouse, rat, rabbit, and guinea pig). In some embodiments a subject is a mammal. A mammal can be any age or at any stage of development (e.g., an adult (e.g., 18, 19, 20 or 21 years and older), a senior adult (e.g., over the age of 55, over the age of 60, or over the age of 65 years), a teen (e.g., age 12 to 19 yrs.), child (e.g., age 1 to 12 yrs.), infant (e.g., from birth to 1 yr.), or a mammal in utero). A mammal can be male or female. In some embodiments, a subject is a human.

In some embodiments, a subject has, is suspected of having, or is at risk of developing, a motor neuron disease. In some embodiments the motor neuron disease is ALS. In other embodiment, the motor neuron disease is PLS. In some embodiments, a subject who has a motor neuron disease is a subject diagnosed as having a motor neuron disease by a medical professional (e.g., a medical doctor) based on, for example the presence of one or more diagnostic symptoms and/or the results of one or more standardized diagnostic test results. A subject suspected of having a motor neuron disease is a subject not yet diagnosed as having a motor neuron disease by a medical professional. In some embodiments, a subject suspected of having a motor neuron disease may display one or more symptoms of a motor neuron disease, which symptoms are not conclusive evidence that the subject has a motor neuron disease. In some embodiments, a subject who is suspected of a having a motor neuron disease may have one or more symptoms of a motor neuron disease, but is not diagnosed as having any one particular motor neuron disease because there is not enough data to indicate conclusively that the subject has a particular motor neuron disease. In some embodiment, a subject who is suspected of having a motor neuron disease is a subject suspected of having ALS and/or PLS, but is not diagnosed as having ALS and/or PLS by a medical professional.

In some embodiments, a subject is determined to be a subject at risk of developing a motor neuron disease by carrying out one or more of the methods described herein. In some embodiments, a subject at risk of developing a motor neuron disease is a subject who is asymptomatic for a motor neuron disease. In some embodiments, a subject at risk of developing a motor neuron disease is a subject having one or more symptoms of a motor neuron disease, which symptoms may be mild or transient in nature. In some embodiments, a subject at risk of developing a motor neuron disease is a subject who is not yet diagnosed as having a motor neuron disease. In some embodiments, a subject at risk of developing a motor neuron disease is a subject suspected of having a motor neuron disease. In additional embodiments, a subject is a healthy individual tested as a preventative screen to rule out the possibility of being at risk of developing a motor neuron disease and to identify the possibility of onset at an early stage before symptoms arise.

In certain embodiments, the presence or absence of ALS and/or PLS in a subject is determined by the methods described herein. In some embodiments, wherein a subject is determined to have ALS and/or PLS that is diagnosed by one or more of the methods described herein, there is further provided a method of treating ALS and/or PLS by administering a suitable treatment to the subject, wherein the ALS and/or PLS, or one or more symptoms thereof are therapeutically treated. In certain embodiments, the methods described herein identify a subject who is at risk of developing ALS and/or PLS. In some embodiments, wherein a subject is identified as at risk of developing ALS and/or PLS by the methods described herein, there is further provided a method of treating that subject at risk of developing ALS and/or PLS by administering a suitable treatment to the subject, wherein development of ALS and/or PLS is prevented or delayed, or wherein one or more symptoms thereof are therapeutically treated. In some embodiments, a method of treating ALS and/or PLS is a method of inhibiting or delaying the onset or progression of ALS and/or PLS, for example in a subject at risk of developing ALS and/or PLS. In some embodiments, a method comprises treating ALS and/or PLS or one or more symptoms thereof. In some embodiments, a method of treating ALS and/or PLS is a method of inhibiting or delaying the onset or progression of one or more symptoms of AL and/or PLS, for example in a subject at risk of developing ALS and/or PLS.

Non-limiting examples of a symptom of a motor neuron disease include a motor deficiency; fatigue (e.g., excessive fatigue); passivity; lethargy; inertia; tremors; ataxia; speaking difficulty (e.g., slurred, thick or irregular speech); muscle cramps (e.g., excessive muscle cramping, not necessarily induced by excessive use or excessive exercise), twitching, atrophy or weakness; shortness of breath; breathing difficulty; writing difficulty; unusual or frequent stiffness or rigidity; loss of fine or gross motor control; slowing of movement; impaired balance; body instability; posture or gait abnormality (e.g., shuffling walk, unsteady or irregular gait); reduced coordination; motor dysfunction; jerky or involuntary body movement; slowed saccadic eye movement; seizures; difficulty chewing, eating, or swallowing; loss of balance; opthalmoparesis or impaired eye movement; impaired eyelid function; involuntary facial muscle contracture; neck dystonia or backward tilt of the head with stiffening of neck muscles; urinary/bowel incontinence; parkinsonism; the like and combinations thereof.

In some embodiments, a method comprises preventing or treating ALS and/or PLS, inhibiting or delaying the onset of, or progression of ALS and/or PLS, or inhibiting, mitigating, reducing or delaying the onset of one or more symptoms of ALS and/or PLS, where the method comprises administering a therapeutically effective amount of a ALS and/or PLS disease drug, non-limiting examples of which include L-serine, ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenibut, imagabalin, atagabalin, 4-methylpregabalin, PD-217,014, Riluzole, Edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine (e.g., an immunogenic amount of an amyloid peptide, or a fragment or variant thereof, with or without an adjuvant), a cholinesterase inhibitor (e.g., donepezil, galantamine or rivastigmine), memantine, an antidepressant, an N-methyl D-aspartate (NMDA) antagonist, an omega-3 fatty acid, curcumin, or a curcumin derivative, vitamin E, a sleep aid (e.g., zolpidem, eszopiclone or zaleplon), an anti-anxiety drug (e.g., lorazepam and clonazepam), an anti-convulsant (e.g., sodium valproate, carbamazepine, or oxcarbazepine), an anti-psychotic (e.g., risperidone, quetiapine or olanzapine), carbidopa-levodopa, amantadine, a dopamine agonists (e.g., pramipexole, ropinirole, rotigotine or Apomorphine), a MAO B inhibitor (e.g., selegiline, rasagiline or safinamide), a Catechol O-methyltransferase (COMT) inhibitors (e.g., entacapone or tolcapone), an anticholinerigic (e.g., benztropine or trihexyphenidyl), the like and combinations thereof. In some embodiments, a subject diagnosed as having ALS, or at risk of developing ALS and/or PLS, is treated by a method comprising administering a therapeutically effective amount of one or more of L-serine, Riluzole, Edaravone, Nusinersen, Onasemnogeme abeparovec-xioi (ZOLGENSMA™), Radicava, Rilutek, Tiglutik, Nuedexta, muscle relaxers (e.g., baclofen, tizanidine, benzodiazepines) or botulinum toxin. In some embodiments, a subject diagnosed as having ALS and/or PLS, or at risk of developing ALS and/or PLS is treated by a method comprising administering a therapeutically effective amount of L-serine.

In some embodiments, a suitable method of treatment comprises administering a therapeutically effective amount of ralitoline, phenytoin, lamotrigine, carbamazepine, lidocaine, tetrodotoxin, Riluzole, Edaravone, Gabapentin, pregabalin, Mirogabalin, gabapentin enacarbil, phenibut, imagabalin, atagabalin, 4-methylpregabalin, PD-217,014, Trihexyphenidyl, amitriptyline, baclofen, diazepam, L-serine, CK-2127107 (reldesemtiv), Nusinersen, Onasemnogeme abeparovec-xioi (ZOLGENSMA™), Radicava, Rilutek, Tiglutik, Nuedexta, the like or combinations thereof. In some embodiments, a suitable method of treatment comprises administering a therapeutically effective amount of L-serine.

### L-Serine

In some embodiments, a subject is administered a therapeutically effective amount of L-serine, a salt, metabolic precursor, derivative or conjugate thereof. In some embodiments, a subject is administered a therapeutically effective amount of free L-serine, or a salt thereof. A therapeutically effective amount of L-serine or free L-serine may be administered as a pharmaceutical composition comprising one or more pharmaceutical excipients, additives, carriers and/or diluents. In some embodiments, a method herein comprises administered a therapeutically effective amount of a composition comprising, consisting of, or consisting essentially of L-serine, a salt, metabolic precursor, derivative or conjugate thereof to a subject. In some embodiments, a method herein comprises administered a therapeutically effective amount of a composition comprising, consisting of, or consisting essentially of free L-serine, or a salt, derivative or conjugate thereof to a subject. In some embodiments, a method herein comprises administering a therapeutically effective amount of a composition comprising, consisting of, or consisting essentially of a polymer of L-serine, or a salt, derivative or conjugate thereof to a subject. In some embodiments, a composition consisting essentially of L-serine, free L-serine, or a salt, a precursor, a derivative or a conjugate thereof excludes proteins or protein fractions comprising less than 100%, 99%, 98%, less than 95%, less than 90%, less than 80%, less than 70%, less than 60%, or less than 50% L-serine (wt/wt). In some embodiments, a composition consisting essentially of L-serine, free L-serine, or a salt, a precursor, a derivative or a conjugate thereof excludes proteins or protein fractions comprising greater than 5%, greater than 10%, greater than 20%, greater than 30%, greater than 40%, greater than 50% or greater than 60% protein (wt/wt), and excludes other components that materially affect the therapeutic efficacy of the L-serine to prevent and/or treat ALS. In some embodiments, a composition consisting essentially of L-serine comprises free L-serine, or a polymer of L-serine having an amino acid content of L-serine of at least 100%, 99%, 98%, 95%, 90%, 85% or at least 80%. In some embodiments, a composition consisting essentially of L-serine excludes creatine, creatine pyruvate, guanidino-acetic acid (GA), glycocyamine, N-amidinoglycine, and salts or esters thereof. In some embodiments, a composition consisting essentially of L-serine is a composition comprising free L-serine at a purity of at least 85%, at least 90%, at least 95%, at least 98%, at least 99% or 100%. In certain embodiments, a composition consisting essentially of L-serine, free L-serine, or a salt, a precursor, derivative or conjugate of L-serine, is a composition that also comprises zinc.

Free L-serine refers to L-serine in the form of a single amino acid monomer, or a salt thereof. In some embodiments, a composition comprises free L-serine at a purity of at least 85%, at least 90%, at least 95%, at least 98%, at least 99% or 100%. In certain embodiments, free L-serine is not covalently bonded to any other amino acid.

In some embodiments, a composition comprising L-serine may exclude other active ingredients. In some embodiments, a composition may exclude proteins containing L-serine. In some embodiments, a composition may exclude proteins having a molecular weight greater than 10 kDa, greater than 20 kDa, greater than 30 kDa or greater than 50 kDa. In some embodiments, a composition may exclude proteins containing less than 99%, 98%, 95%, 92%, 90%, 80%, 70%, 60%, or less than 50% L-serine. In some embodiments, a composition may exclude creatine, or any energy metabolism precursor of creatine, such as guanidino-acetic acid (GA), equivalents thereof, and mixtures thereof.

In certain embodiments, a composition comprises L-serine, non-limiting examples of which include free L-serine, and polymers or polypeptides comprising at least a 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% L-serine by weight or amino acid content. In some embodiments, a polymer of L-serine or a polypeptide comprising L-serine includes between 2 and 50000, between 2 and 500, between 2 and 100, between 2 and 50, between 2 and 20, between 2 and 15, between 2 and 10, between 2 and 9, between 2 and 8, between 2 and 7, between 2 and 6, between 2 and 5, or between 2 and 4 L-serine amino acids linked by covalent bonds. In certain embodiments, a composition comprises L-serine, non-limiting examples of which include a polymer or polypeptide comprising from 20% to 100%, from 30% to 100%, from 35% to 100%, from 40% to 100%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, from 95% to 100%, from 96% to 100%, from 97% to 100%, from 98% to 100%, or from 99% to 100% content of L-serine (wt/wt) or amino acid content (i.e., L-serine monomers/total amino acid monomers).

Non-limiting examples of a salt of L-serine include a sodium salt, potassium salt, calcium salt, magnesium salt, zinc salt, ammonium salt; inorganic salts such as, hydrogen chloride, sodium chloride, potassium chloride, calcium chloride, sodium phosphate, potassium phosphate, and sodium hydrogen carbonate; organic salts such as, sodium citrate, citrate, acetate, and the like. In certain embodiments, a composition comprises L-serine as an alkylated L-serine, such as L-serine with an alkyl group, or e.g., an alkyl comprising 1-20 carbon atoms. In certain embodiments, a derivative of L-serine includes an L-serine ester, an L-serine di-ester, a phosphate ester of L-serine, or a sulfate or sulfonate ester of L-serine. Non-limiting examples of a conjugate of L-serine includes a pegylated L-serine (e.g., an L-serine comprising one or more polyethylene glycol (PEG) moieties), and a lipidated L-serine. Non-limiting example of a precursor of L-serine include L-phosphoserine.

Non-limiting examples of a precursor of L-serine include a pro-form of L-serine that is broken down into L-serine monomers by the digestive system of a subject. In some embodiments, L-serine or a conjugate thereof consists of a slow-release version. In some embodiments a derivative of L-serine is conjugated to a different molecule forming a prodrug from which L-serine is released after crossing the blood/brain barrier.

In some embodiments, a composition consisting essentially of L-serine may comprise some amount of D-serine. For example, a composition consisting essentially of L-serine may include a small amount of D-serine, for example, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1% D-serine by weight (e.g., wt/wt) or amino acid content (e.g., L-serine/total amino acid content). For example, a composition may include from 0.001% to 30%, from 0.005% to 30%, from 0.1% to 30%, from 1% to 30%, from 2% to 30%, from 3% to 30%, from 4% to 30%, from 5% to 30%, from 6% to 30%, from 7% to 30%, from 8% to 30%, from 9% to 30%, from 10% to 30%, from 0.001% to 20%, from 0.005% to 0%, from 0.1% to 20%, from 1% to 20%, from 2% to 20%, from 3% to 20%, from 4% to 20%, from 5% to 20%, from 6% to 20%, from 7% to 20%, from 8% to 20%, from 9% to 20%, or from 10% to 20% D-serine. In some embodiments, a composition comprising or consisting essentially of L-serine, does not comprise a substantial amount of D-serine. In some embodiments, a composition comprising or consisting essentially of L-serine, does not contain D-serine.

### Methods Using Circulating Bodily Fluid

In some embodiments, a method presented herein detects or determines an amount of one or more miRNAs associated with ALS and/or PLS from circulating bodily fluid, for example, from a subject's blood, without isolating exosomes. The inventors' prior efforts carried out methods using exosomes, where the isolation, separation, identification, etc. of various miRNAs can be time consuming and cumbersome. While the previous method was effective in identifying panels of miRNAs associated with a motor neuron disorder such as ALS and/or PLS, using exosomes as a commercial diagnostic poses some difficulties, including difficulty in automation, labor and time intensity, etc. It also would not have been known or appreciated that the same or all of miRNAs identified using neural-derived exosomes could also be detected in a subject's circulating body fluid without isolating and analyzing exosomes, nor would it have been expected that a diagnostic test using the same or all of these miRNAs from circulating bodily fluid would be as accurate or sensitive as a diagnostic test using neural-derived exosomes.

The inventors unexpectedly discovered that the same miRNA can be found in circulating blood plasma without the neural-enriched or derived extracellular vesicles (NEE, exosomes) isolation and analysis steps, and that the same miRNA are more concentrated, providing a better signal for diagnostic applications. In some embodiments, a method herein comprises determining the presence or amount of one or more miRNAs selected from one or more of miR-199a-3p, miR-4454, miR-10b-5p, miR-151a-5p, miR-199a-5p, miR-151a-3p, miR-146a-5p, and/or miR-29b-3p from a subject's circulating bodily fluid. In some embodiments, a method herein comprises determining the presence or amount of one or more miRNAs selected from one or more of miR-199a-3p, miR-4454, miR-10b-5p, miR-151a-5p, miR-199a-5p, miR-151a-3p, miR-146a-5p, and/or miR-29b-3p from a subject's circulating bodily fluid prepared from a sample obtained from a subject that has, or is suspected of having, a motor neuron disease. In some embodiments, a method herein comprises determining the presence or amount of one or more miRNAs selected from one or more of miR-199a-3p, miR-4454, miR-10b-5p, miR-151a-5p, miR-199a-5p, miR-151a-3p, miR-146a-5p, and/or miR-29b-3p from a subject's circulating bodily fluid prepared from a sample obtained from a control subject. Non-limiting examples of a control subject include healthy subjects, a subject that does not have a motor neuron disease and/or a subject that is not suspected of having a motor neuron disease.

A feature of an embodiment includes a method of identifying a subject who has, or is at risk of developing a motor neuron disease such as ALS and/or PLS, the method comprising: (a) determining a presence or amount of one or more micro-RNAs (miRNAs) selected from the group consisting of miR-199a-3p, miR-4454, miR-10b-5p, miR-151a-5p, miR-199a-5p, miR-151a-3p, miR-146a-5p, and/or miR-29b-3p from a subject's circulating bodily fluid without determining a presence or amount from a subject's neural-derived exosomes, and determining if the subject has, or is at risk of developing the motor neuron disease such as ALS and/or PLS, according to the presence or amount of the one or more miRNAs in the sample. In certain embodiments, the method comprises determining the presence or amount of two or more, three or more, four or more, five or more, six or more, seven or more, or all eight of the miRNAs selected from the group consisting of miR-199a-3p, miR-4454, miR-10b-5p, miR-151a-5p, miR-199a-5p, miR-151a-3p, miR-146a-5p, and miR-29b-3p. In certain embodiments, the presence of four or more, five or more, six or more, seven or more or all eight of the miRNAs selected from the group consisting of miR-199a-3p, miR-4454, miR-10b-5p, miR-151a-5p, miR-199a-5p, miR-151a-3p, miR-146a-5p, and miR-29b-3p in, on or within a subject's circulating bodily fluid obtained from a subject, indicates that the subject has or is at risk of having a motor neuron disease such as ALS and/or PLS.

In some embodiments, a method herein comprises comparing an amount of one or more miRNAs in circulating bodily fluid obtained from a sample from a control subject (e.g., a subject known to be free of a motor neuron disease) to an amount of one or more miRNAs in circulating bodily fluid obtained from a sample from a test subject (e.g., a subject suspected of having a motor neuron disease). In some embodiments, the presence or absence of a motor neuron disease in a test subject is determined according to such a comparison. In some embodiments, a subject at risk of developing a motor neuron disease is identified according to such a comparison. In some embodiments, a comparison determines that the amount of one or more miRNAs associated with neural-derived exosomes obtained from a first (or test) subject are significantly lower, or significantly higher than those obtained from a control subject.

The term “significantly” as used throughout refers to a statically significant difference that can be determined using a suitable statistical method (e.g., a t-test). In some embodiments, a comparison determines that the amount of one or more miRNAs obtained from a sample of a first subject's circulating bodily fluid are significantly higher than those of a control subject, thereby indicating that the first subject has a neurogenerative disease or has a high statistical likelihood of developing a neurogenerative disease.

In some embodiments, a comparison determines that the amount of one or more miRNAs obtained from a sample of a subject's circulating bodily fluid is from about 1.1-fold to about 50 fold higher or lower than a baseline amount of such one or more miRNAs, thereby indicating that the subject has a neurogenerative disease or has a statistical likelihood of developing a neurogenerative disease (i.e., is “at risk” of developing, e.g., a motor neuron disease such as, e.g., ALS and/or PLS). In some embodiments, a comparison determines that the amount of one or more miRNAs obtained from a sample of a first subject's circulating bodily fluid is from about 1.5 to about 50 fold higher or lower, or any value or ranges of values therebetween, than the baseline amount of such one or more miRNAs, thereby indicating that the subject has a neurogenerative disease or has a statistical likelihood of developing a neurogenerative disease (i.e., is “at risk” of developing, e.g., a motor neuron disease such as, e.g., ALS and/or PLS). Throughout this description, the disclosure of range, such as from about 5 to about 10, includes any value between 5 and 10, as well as any range of values between 5 and 10 such as 5-9, or 6-10, or 6-9, or 5-8, or 5, 6, 7, 8, 9, or 10, etc.

In some embodiments, an amount of miR-146a-5p that is at least 1.4, at least 1.5, at least 1.6, at least 1.7, at least 1.8, or at least 1.9 fold higher than a base line amount of miR-146a-5p in circulating body fluid obtained from a subject means that the subject has or is at risk of developing a motor neuron disease, specifically, ALS and/or PLS. In some embodiments, an amount of miR-199a-3p that is at least 1.4, at least 1.5, at least 1.6, at least 1.9, at least 2.0, or at least 2.1 fold higher that a base line amount of miR-199a-3p in in circulating body fluid obtained from a subject means that subject has or is at risk of developing a motor neuron disease, specifically, ALS and/or PLS.

In some embodiments, an amount of miR-4454 that is at least 1.9, at least 2.1, at least 2.3, at least 2.6, or at least 2.8 fold lower that a base line amount of miR-4454 in in circulating body fluid obtained from a subject means that subject has or is at risk of developing a motor neuron disease, specifically, ALS and/or PLS. In some embodiments, an amount of miR-10b-5p that is at least 6, at least 7, at least 8, or at least 10-fold lower that a base line amount of miR-10b-5p in circulating body fluid obtained from a subject means that the subject has or is at risk of developing a motor neuron disease, specifically, ALS. and/or PLS

In some embodiments, an amount of miR-29b-3p that is at least 1.0, at least 1.1, at least 1.2, or at least 1.3-fold lower that a base line amount of miR-29b-3p in circulating body fluid obtained from a subject means that the subject has or is at risk of developing a motor neuron disease, specifically, ALS and/or PLS. In some embodiments, an amount of miR-151a-3p that is at least 1.4, at least 1.6, at least 1.8, at least 1.9, at least 2.1, or at least 2.5-fold higher that a base line amount of miR-151a-3p in circulating body fluid obtained from a subject means that the subject has or is at risk of developing a motor neuron disease, specifically, ALS and/or PLS.

In some embodiments, an amount of miR-151a-5p that is at least 1.4, at least 1.5, at least 1.6, at least 1.8, or at least 1.9-fold higher that a base line amount of miR-151a-5p in circulating body fluid obtained from a subject means that the subject has or is at risk of developing a motor neuron disease, specifically, ALS and/or PLS. In some embodiments, an amount of miR-199a-5p that is at least 2.0, at least 2.3, at least 2.5, at least 2.6, or at least 2.8-fold higher that a base line amount of miR-199a-5p in circulating body fluid obtained from a subject means that the subject has or is at risk of developing a motor neuron disease, specifically, ALS. and/or PLS

In some embodiments, such a comparison determines that the amount of one or more of the miRNAs: miR-199a-3p, miR-4454, miR-10b-5p, miR-151a-5p, miR-199a-5p, miR-151a-3p, miR-146a-5p, and miR-29b-3p; identified in circulating bodily fluid obtained from a subject, is from about 1.1-fold to about 75-fold higher or lower than the baseline amount of such one or more miRNAs, thereby indicating that the subject has a motor neuron disease or has a statistical likelihood of developing a motor neuron disease (i.e., is “at risk” of developing, e.g., a motor neuron disease such as, e.g., ALS and/or PLS).

The term “baseline amount” as used herein refers to an average, mean, or absolute amount of one or more miRNAs obtained from circulating bodily fluid obtained from one or more suitable control subjects. For example, a control subject can be a subject that does not have a motor neuron disease. In certain embodiments, a control subject is a subject who does not have ALS and/or PLS. Typically such healthy subjects are young adults (e.g., within the ages of 18-30) that show no signs or symptoms of a motor neuron disease and/or have no family history of a motor neuron disease.

In some embodiments, a comparison determines that the amount of one or more miRNAs present in circulating bodily fluid obtained from a first subject is from about 1.1-fold to about 25-fold higher or lower than the amount of such one or more miRNAs present in circulating bodily fluid obtained from a control subject, thereby indicating that the first subject has a motor neuron disease or has a statistical likelihood of developing a motor neuron disease (i.e., is “at risk” of developing, e.g., a motor neuron disease such as, e.g., ALS and/or PLS). In some embodiments, such a comparison determines that the amount of one or more of the miRNAs: miR-199a-3p, miR-4454, miR-10b-5p, miR-151a-5p, miR-199a-5p, miR-151a-3p, miR-146a-5p, and miR-29b-3p present in circulating bodily fluid obtained from a first subject is from about 1.1-fold to about 25-fold higher or lower than the amount of such one or more miRNAs present in circulating bodily fluid obtained from a control subject, thereby indicating that the first subject has a motor neuron disease or has a statistical likelihood of developing a motor neuron disease (i.e., is “at risk” of developing, e.g., a motor neuron disease such as, e.g., ALS and/or PLS).

In some embodiments, the presence or absence of a motor neuron disease in a subject is determined according to an amount of one or more miRNAs that is associated with circulating bodily fluid obtained from a subject. In some embodiments, an amount of at least 0.01 mean normalized copy number/μl of miRNA, at least 0.05 mean normalized copy number/μl of miRNA, at least 0.1 mean normalized copy number/μl of miRNA, at least 0.5 mean normalized copy number/μl of miRNA, at least 1 mean normalized copy number/μl of miRNA, at least 3 mean normalized copy number/μl of miRNA, at least 5 mean normalized copy number/μl of miRNA, at least 10 mean normalized copy number/μl of miRNA, or more of miRNA that is associated with circulating bodily fluid obtained from a subject indicates that the subject has a motor neuron disease or has a statistical likelihood (i.e., is “at risk”) of developing a motor neuron disease. In other embodiments, an amount of at least 50 pg/μL of miRNA, at least 100 pg/μL of miRNA, at least 150 pg/μL of miRNA, at least 200 pg/μL of miRNA, at least 250 pg/μL of miRNA, at least 300 pg/μL of miRNA, at least 350 pg/μL of miRNA, at least 400 pg/μL of miRNA, at least 450 pg/μL of miRNA, at least 500 pg/μL of miRNA, at least 550 pg/μL of miRNA, at least 600 pg/μL of miRNA, at least 650 pg/μL of miRNA, at least 700 pg/μL of miRNA, at least 750 pg/μL of miRNA, at least 800 pg/μL of miRNA, at least 850 pg/u L of miRNA, at least 900 pg/μL of miRNA, at least 950 pg/μL of miRNA, at least 1000 pg/μL of miRNA, or more of miRNA that is associated with circulating bodily fluid obtained from a subject indicates that the subject has a motor neuron disease or has a statistical likelihood (i.e., is “at risk”) of developing a motor neuron disease. In an embodiment, the one or more miRNAs are selected from the group consisting of miR-199a-3p, miR-4454, miR-10b-5p, miR-151a-5p, miR-199a-5p, miR-151a-3p, miR-146a-5p, and miR-29b-3p.

In some embodiments, methods are provided for monitoring the progression of a motor neuron disease in a subject. In some embodiments, such a method comprises a) obtaining a circulating bodily fluid sample from the subject; (b) determining an amount of one or more miRNAs present in the circulating bodily fluid, such as one or more miRNAs selected from the group consisting of miR-199a-3p, miR-4454, miR-10b-5p, miR-151a-5p, miR-199a-5p, miR-151a-3p, miR-146a-5p, and miR-29b-3p, without determining an amount from a subject's neural-derived exosomes; and (c) comparing the amount of the one or more miRNAs determined in step (b) to a baseline amount of the one or more miRNAs; and (d) determining the progression or lack of progression of the motor neuron disease based on the comparison in (c).

In some embodiments, methods are provided for monitoring a response to treatment of a motor neuron disease in a subject. In some embodiments, such a method comprises a) obtaining a circulating bodily fluid sample from the subject; (b) determining an amount of one or more miRNAs present in the circulating bodily fluid, such as one or more miRNAs selected from the group consisting of miR-199a-3p, miR-4454, miR-10b-5p, miR-151a-5p, miR-199a-5p, miR-151a-3p, miR-146a-5p, and miR-29b-3p without determining an amount from a subject's neural-derived exosomes; (c) comparing the amount of the one or more miRNAs determined in step (b) to a baseline amount of the one or more miRNAs; and (d) monitoring the response to treatment based on the comparison in (c). In some embodiments, a difference between an amount of one or more miRNAs from the circulating bodily fluid obtained from a subject after treatment of the subject has commenced and a baseline amount that is less than the difference obtained at an earlier point in time indicates that the subject has responded favorably to the treatment. In some embodiments, a difference between an amount of one or more miRNAs from the circulating bodily fluid obtained from a subject and a baseline amount that is greater than the difference obtained at an earlier point in time indicates that the subject has not responded favorably to the treatment.

In some embodiments, the baseline amount may be an amount considered ‘normal’ for the particular miRNA (e.g., an average amount for age-matched individuals not diagnosed with the motor neuron disease), or the baseline amount may be a historical reference amount for the particular subject (e.g., a baseline amount that was obtained from a circulating bodily fluid sample derived from the same subject, but at an earlier point in time). Quantitative baseline amounts that are determined contemporaneously (e.g., a reference value that is derived from a pool of samples including the sample being tested) are also contemplated. Accordingly, in some embodiments, methods are provided for monitoring progression of a motor neuron disease in a subject by obtaining a quantitative measured amount for one or more miRNAs present in circulating bodily fluid obtained from a sample and comparing such measured value to a baseline amount. In some embodiments, a difference between an amount of one or more miRNAs from circulating bodily fluid obtained from a subject and a baseline amount that is less than the difference obtained at an earlier point in time indicates that the progression of the disease has diminished. In some embodiments, a difference between an amount of one or more miRNAs from circulating bodily fluid obtained from a subject and a baseline amount that is greater than the difference obtained at an earlier point in time indicates that the progression of the disease has increased.

### Administration

Any suitable method of administering a treatment or drug to a subject can be used. Any suitable formulation and/or route of administration can be used for administration of a treatment or drug disclosed herein (e.g., see Fingl et al. 1975, in “The Pharmacological Basis of Therapeutics”, which is incorporated herein by reference in its entirety). A suitable formulation and/or route of administration can be chosen by a medical professional (e.g., a physician) in view of, for example, a subject's disease, condition, symptoms, weight, age, and/or general health. Non-limiting examples of routes of administration include topical or local (e.g., transdermally or cutaneously, (e.g., on the skin or epidermis), in or on the eye, intranasally, transmucosally, in the car, inside the car (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments administering a drug to a subject comprises providing the drug to the subject, for example for self-administration or for administration to the subject by another (e.g., by a non-medical professional). As another example, a drug can be provided as an instruction written by a medical practitioner that authorizes a patient to be provided a drug or treatment described herein (e.g., a prescription). In yet another example, a drug can be provided to a subject where the subject self-administers a composition orally, intravenously or by way of an inhaler, for example. Alternately, one can administer a drug in a local rather than systemic manner, for example, via direct application to the skin, mucous membrane or region of interest for treating, including using a depot or sustained release formulation. In certain embodiments a drug is administered alone (e.g., as a single active ingredient (AI) or, e.g., as a single active pharmaceutical ingredient (API)). In other embodiments, a drug is administered in combination with one or more additional AIs/APIs, for example, as two separate compositions or as a single composition where the one or more additional AIs/APIs are mixed or formulated together with a drug in a pharmaceutical composition.

In some embodiments, an amount of a motor neuron disease drug administered to a subject is a therapeutically effective amount. In some embodiments, a therapeutically effective amount of a drug is an amount needed to obtain an effective therapeutic outcome. In certain embodiments, a therapeutically effective amount of a drug is an amount sufficient to treat, reduce the severity of, inhibit or delay the onset of, mitigate and/or alleviate one or more symptoms of a motor neuron disease. Determination of a therapeutically effective amount is well within the capability of those skilled in the art, especially in light of the detailed disclosure provided herein.

In certain embodiments, a therapeutically effective amount is an amount high enough to provide an effective therapeutic effect (e.g., a beneficial therapeutic effect) and an amount low enough to minimize unwanted adverse reactions. Accordingly, in certain embodiments, a therapeutically effective amount of a drug may vary from subject to subject, often depending on age, weight, general health condition of a subject, severity of a condition being treated and/or a particular combination of drugs administered to a subject. Thus, in some embodiments, a therapeutically effective amount is determined empirically. Accordingly, in certain embodiments, a therapeutically effective amount of a drug that is administered to a subject can be determined by one of ordinary skill in the art based on amounts found effective in animal or clinical studies, a physician's experience, and/or suggested dose ranges or dosing guidelines.

In certain embodiments, a therapeutically effective amount of L-serine or a composition disclosed herein comprises one or more doses (administered to a subject) comprising at least 0.1 mg/kg, at least 5 mg/kg, at least 10 mg/kg, at least 15 mg/kg, at least 20 mg/kg, at least 25 mg/kg, at least 50 mg/kg, at least 100 mg/kg, at least 250 mg/kg, at least 500 mg/kg, at least 1000 mg/kg, at least 5000 mg/kg, or at least 7500 mg/kg of L-serine, or a salt, a precursor, derivative or conjugate thereof, per kg body weight of a subject.

In some embodiments administering a therapeutically effective amount of a motor neuron disease drug or composition disclosed herein comprises administering a suitable dose hourly, every two hours, every 4 hours, every 6 hours, every 8 hours, or every 12 hours. In certain embodiments, a motor neuron disease drug can be administered at least one, at least two, at least three, at least four, at least five, or at least six times per day, e.g., 1 to 12 times per day, 1 to 8 times per day, or 1 to 4 times per day per day. In certain embodiments, a motor neuron disease drug disclosed herein can be administered once, twice, 3 times, 4 times, 5 times, 6 times, 7 times, 8 times, 9 times, 10 times, 11 times, or 12 times per day. A motor neuron disease drug may be administered in a single dosage form or one or more dosage forms. A daily dose can be achieved in the form of a single dose or in the form of a plurality of partial doses.

A motor neuron disease drug disclosed herein can be administered on a daily basis or on a schedule containing days where dosing does not take place. For example, dosing may take place every other day, or dosing may take place for 2, 3, 4, or 5 consecutive days of a week, then be followed by from 1 to 5 non-dosing days.

A motor neuron disease drug can be administered for at least a day, at least two days, at least three days, at least four days, at least five days, at least a week, at least two weeks, at least three weeks, at least a month, at least two months, at least three months, at least six months, at least a year, at least two years, or more, or for any extended duration to further improve, maintain, or retain therapeutic efficacy. In certain embodiments, a motor neuron disease drug is administered for a duration of 1 week to 10 years or more. In some embodiments administering a therapeutically effective amount of a drug, or a pharmaceutical composition comprising a drug, comprises administering a suitable dose at a frequency or interval as needed to obtain an effective therapeutic outcome. In some embodiments administering a therapeutically effective amount of a drug or a pharmaceutical composition disclosed herein comprises administering a suitable dose hourly, every two hours, every 4 hours, every 6 hours, three times a day, twice a day, once a day, six times a week, five times a week, four times a week, three times a week, twice a week, weekly, at combinations thereof, and/or at regular or irregular intervals thereof, and/or simply at a frequency or interval as needed or recommended by a medical professional. In some embodiments, a therapeutically effective amount of a drug or a pharmaceutical composition comprising a therapeutically effective amount of drug is administered continuously by, for example by intravenous administration.

### Kits

In some aspects, presented herein is one or more kits useful in carrying out a method of identifying a subject who has, or is at risk of developing a motor neuron disease that includes one or more multi-well plates, with each well contains one or more miRNA primers selected from the group consisting of miR-199a-3p, miR-4454, miR-10b-5p, miR-151a-5p, miR-199a-5p, miR-151a-3p, miR-146a-5p, miR-29b-3p, and a mechanism to calculate the likelihood of a positive or negative diagnosis, and/or probability of having or not having a neurological disease. In one embodiment, the kit includes two well-plates in which one well plate includes each well containing one or more miRNA primers selected from the list above, and the other well plate includes a Quality Control (QC)/spike-in/calibration plate in which one or more wells contain QC primers, no-template controls, and/or spike-in primers designed to monitor the success of the method. In another embodiment, the kit includes one well-plate in which the wells include one or more miRNA primers selected from the list above, QC primers, no-template controls, and/or spike-in primers designed to monitor the success of the method. In a further embodiment, the mechanism to calculate the likelihood of a positive or negative diagnosis, and/or probability of having or not having a neurological disease includes software configured to carry out the calculation using random forest machine learning algorithms and/or logistic regression algorithms developed from machine learning classification of known disease and healthy control plasma samples.

The well-plates useful in the kits described herein may include, for example, qPCR plates of varying sizes, including 48, 96, and 384-well plates. Each of the respective wells can pre-loaded with one or more of the miRNA primers selected from miR-199a-3p, miR-4454, miR-10b-5p, miR-151a-5p, miR-199a-5p, miR-151a-3p, miR-146a-5p, miR-29b-3p, as well as QC primers, no-template controls, and/or spike-ins. Particularly preferred QC primers, no-template controls, and/or spike-in primers that may be used in the kit(s) described herein are described in more detail in the examples below. In one embodiment, a kit may contain two 96-well plates suitable for 25 μL reactions in which one of the well-plates contains wells filled with one or more of the miRNA primers selected from miR-199a-3p, miR-4454, miR-10b-5p, miR-151a-5p, miR-199a-5p, miR-151a-3p, miR-146a-5p, miR-29b-3p, and the other well-plates contains wells filed with QC primers, no-template controls, and/or spike-in primers. In another embodiment, the kit includes a 384-well plate suitable for 10 μL reactions, in which all of the miRNA, QC primers, no-template controls, and/or spike-in primers are included on the same plate.

The kits are particularly suited for automated pipetting using a liquid handling robot (e.g., an Opentrons OT-2 robot) for sample loading into the respective wells in the plate(s) of the respective kit(s) that are preloaded with the reagents discussed above. Operators supply the RNA/cDNA. The kits can be designed for use in any analyzer capable of carrying out qPCR analysis on the circulating bodily fluid samples added to the respective well-plates, and indicating a presence and/or amount of one or more of the miRNA primers selected from miR-199a-3p, miR-4454, miR-10b-5p, miR-151a-5p, miR-199a-5p, miR-151a-3p, miR-146a-5p, miR-29b-3p. The method of diagnosis can be carried out using the information generated regarding the presence and/or amount of one ore more of the miRNAs selected from miR-199a-3p, miR-4454, miR-10b-5p, miR-151a-5p, miR-199a-5p, miR-151a-3p, miR-146a-5p, miR-29b-3p.

The diagnostic method preferably is carried out on-site using a mechanism for calculating the likelihood of a positive or negative diagnosis, and/or probability of having or not having a neurological disease. A preferred device for conducting the qPCR includes the use of a convention qPCR real time diagnostic system, such as those available as BioRad CFX series, Applied Biosystems, Edvotek, MSE PRO series, Benchmark Scientific, and the like. The data from the qPCR system can be fed to a computing apparatus configured to calculate the likelihood of a positive or negative diagnosis, and/or probability of having or not having a neurological disease.

The calculation may be performed using random forest machine learning algorithms and/or logistic regression algorithms developed from machine learning classification of known disease and healthy control plasma samples. Random forest machine learning algorithms are well-known and described in the art, and typically rely on random forest algorithms that have three main hyperparameters, node size, number of trees, and number of features sampled, that are predetermined. Logistic regression machine learning algorithms also are well known in the art and are used to accomplish binary classification tasks by predicting the probability of an outcome, event, or observation. Those skilled in the art are capable of using random forest machine learning algorithms and/or logistic regression algorithms to develop software or a protocol for carrying out the method of identifying a subject who has, or is at risk of developing a motor neuron disease (ALS and/or PLS) using the presence or absence of two more of miRNA selected from miR-199a-3p, miR-4454, miR-10b-5p, miR-151a-5p, miR-199a-5p, miR-151a-3p, miR-146a-5p, miR-29b-3p. The software can be provided with one or more kit(s) described herein the first time the purchaser acquires a kit, and loaded onto a computing device integral to, incorporated into, or in communication with a device for conducting qPCR.

### EXAMPLES

The Examples of U.S. Patent Application Publication No. 2021/0164051 are incorporated herein by reference in their entirety. The Examples of our earlier application provide detailed information regarding the use of neural derived exosomes to determine the presence or amount of one or more, or combinations of certain miRNA associated with the neural derived exosomes. These Examples provide additional information regarding the determination of whether a distinct difference in the presence and/or amounts of one or more, or combinations of certain miRNA associated with the neural derived exosomes is found between healthy, or control, subjects, and subjects known to have a motor neuron disease.

### Example 1—An miRNA Fingerprint of miRNA for Amyotrophic Lateral Sclerosis/Motor Neuron Disease Using Neural-Derived Exosomes

Using retrospective blood samples from previously identified ALS, primary lateral sclerosis, Parkinson's disease patients, and healthy controls, an eight-miRNA fingerprint was evaluated as an aid to diagnosing ALS and/or PLS.

Blood plasma from 119 ALS, 42 primary lateral sclerosis, 20 Parkinson's disease patients, and 150 healthy controls were evaluated for miRNAs contained within neural enriched extracellular vesicles (See Table 1 for study cohort characteristics). Primary lateral sclerosis is considered an ALS-mimic and Parkinson's disease was also included for comparison as aneurological control. Ghasemi, “M. Amyotrophic lateral sclerosis mimic syndromes,” Iran J Neurol. 15 (2): 85-91 (2016). The sample number was limited by the availability of samples. Sample inclusion eligibility was based on diagnosis for ALS, primary lateral sclerosis, or Parkinson's disease, and the absence of a neurological diagnosis for healthy control samples.

100 ALS blood samples, collected across the USA between 2017-2018, were accessed through the USA National ALS Biorepository maintained by the Centers for Disease Control and Prevention, and the Agency for Toxic Substances and Disease Registry (CDC, Advarra IRB Pro00053269). Nineteen ALS blood samples came from a Phase IIa clinical trial (NCT03580616, Dartmouth Institutional Review Board D18095, collected between 2019-2022). 42 blood plasma samples were used from the multi-site primary lateral sclerosis natural history study by Dr. Hiroshi Mitsumoto (Approved by the Columbia University Institutional Review Board (IRB), and by the IRB at each individual site; samples were collected in 2021-2023 following the protocols set forth in Mitsumoto et al., “Primary lateral sclerosis natural history study-planning, designing, and early enrollment,” Amyotrophic Lateral Sclerosis and Frontotemporal Degeneration, 24 (5-6): 394-404 (2023)).

Primary lateral sclerosis was characterized clinically by pure upper motor neuron dysfunction, however, the time since symptom onset is widely accepted by investigators as part of the diagnostic criteria. Samples used in this study fell into three categories: (1) “early PLS” was defined as having upper motor neuron signs present for less than two years after symptom onset (n=4); (2) “probable PLS” was defined as having symptom onset between two to four years (n=18); and (3) patients with a disease duration longer than 4 years since symptom onset were defined as “definite PLS” (n=19). However, no patients were admitted with a disease duration more than 15 years.

20 Parkinson's disease blood plasma samples were obtained from Precision for Medicine (Bethesda, MD), collected across the USA between 2018 and 2020 (Norton, MA, CR00425931). The 150 controls, without known neurological disease, were sourced from Innovative Research Inc (Novi, MI, FDA Approval, #3003372368, collected across the USA prior to 2022) and Precision for Medicine (Norton, MA, CR00425931, collected across the USA from 2020-2022) and were considered to be healthy controls. Selection of these healthy controls were chosen to match the gender and age characteristics of the CDC ALS cohort where possible. Informed consent from all participants was obtained through the respective institutions involved. Since this study used only de-identified participant data, Advarra IRB (Pro00053269) determined that it “does not meet the DHHS definition of human subjects research under 45 CFR 46 and, therefore, does not require IRB oversight.” During the laboratory analyses, researchers were blinded as to the status of the samples and whether they represented disease or control patients.

Blood samples were collected in K₂EDTA tubes and because the samples were obtained from different institutions, plasma processing protocols varied. Samples were blinded before being processed and analyzed. The following methods were used to analyze the samples.

### Extracellular Vesicle Isolation

Extracellular Vesicles (EV) were processed as reported in prior studies using polymer-based precipitation and immunoassay purification: Banack S A, et al., “An miRNA fingerprint using neural-enriched extracellular vesicles from blood plasma: Towards a biomarker for amyotrophic lateral sclerosis/motor neuron disease,” Open Biology, 10 (6): 200116 (2020); and Banack S A, et al., “miRNA extracted from extracellular vesicles is a robust biomarker of amyotrophic lateral sclerosis,” J. Neurol Sci., 442:120396 (2022). Characterization of EVs and differences between total EV collection, neural enriched EV, and the total minus neural enriched extracellular vesicles fraction has been previously published: Banack (2020); and Dunlop R A, et al., “LICAM immunocapture generates a unique extracellular vesicle population with a reproducible miRNA fingerprint,” RNA biology, 20 (1): 140-148 (2023).

### RNA Extraction

Previous studies have described extraction of total RNA retaining short RNA species. We conducted extraction according to the instructions in Qiagen RNeasy Midi Kit Part 2: RNA isolation. We added spike-ins for monitoring RNA extraction efficiency (UniSp2, UniSp4, UniSp5, available as part of the RNA Spike-in Kit, For RT, (Qiagen, cat. no. 339390) at 1 μL per 700 μL lysis buffer (See Dunlop et al., “A comparison of the efficiency of RNA extraction from extracellular vesicles using the Qiagen RNeasy MinElute versus Enzymax LLC RNA Tini Spin columns and qPCR of miRNA,” Biol Methods Protoc., 6 (1): 1-9 (2021) for a detailed modified protocol).

cDNA synthesis

cDNA were synthesized using the miRCURY LNA RT Kit (Qiagen, cat. no. 339340) according to the manufacturer's instructions, and as described previously in Dunlop (2021) above. Modifications included the addition of 1 μL of a spike-in control mix containing UniSp6 and Caenorhabditis elegans cel-miR-39-3p to each cDNA reaction to monitor cDNA synthesis (reverse transcription) efficiency. UniSp6 is provided in the miRCURY LNA RT kit, and cel-miR-39-3p is included in the RNA Spike-in Kit, For RT. 4 μL of total RNA was used rather than 2 μL as recommended by the manufacturer, since it was previously determined that 4 μL returns a more robust signal. Each 10 μL cDNA synthesis reaction was conducted in duplicate and pooled them to create a total of 20 μL cDNA. Prior to storage at −20° C., we aliquoted cDNA into 3 μL aliquots to avoid multiple freeze/thaw cycles.

Real-Time Quantitative PCR (qPCR) Quality Control

Following cDNA synthesis, quality control (QC) qPCR was carried out for every sample on eleven QC miRNA targets. To determine RNA extraction efficiency, the Cqs for the spike-ins UniSp2, UniSp4, and UniSp5 were measured. To determine if reverse transcription (RT) (cDNA synthesis) proceeded without inhibition, the spike-ins UniSp6 and cel-miR-39-3p were measured. Sample signal is a measure of the amount of cDNA in each sample and determines whether samples are suitable for downstream analysis. For determination of sample signal, six endogenous miRNAs: miR-142-3p, miR-451a, miR-23a-3p, miR-30c-5p, miR-103a-3p, and miR-191-5p were measured. Matias-Garcia P R, et al., “Impact of long-term storage and freeze-thawing on eight circulating microRNAs in plasma samples,” PLOS ONE, 15 (1): e0227648 (2020).

qPCR of miRNA Using SYBR Green

qPCR for all QC, target and reference miRNA was conducted using Qiagen miRCURY LNA miRNA SYBR PCR Assays (cat. no. 339306) and the miRCURY LNA™ SYBR Green PCR Kit (Qiagen, cat. no. 339347) according to the manufacturer's instructions, and as described previously (Dunlop (2021)). cDNA 1/30 was diluted into nuclease-free water and 3 μL in each 10 μL were used in each qPCR reaction. The pipetting of master mix (7 μL) and sampling (3 μL) into 384-well plates was automated using the Opentrons OT-2 liquid-handling robot using a custom protocol written in Python. qPCR was conducted on the Bio-Rad CFX Opus 384 in 384-well plates (Bio-Rad Hard Shell PCR Plates, thin-wall, cat. no. HSP3805). Data in Bio-Rad CFX Maestro version 2.3 were acquired after 40 cycles. Each plate contained triplicate inter-plate calibrators (TATAA IPC, TATAA Biocenter, cat. no. IPC250S) pipetted into the same wells on every plate, to enable comparison of samples run in January 2023 with samples run in August 2023. An IPC correction factor was applied to all samples to enable cross-plate comparison. A melt curve and triplicate no-template controls in each assay were included to check for primer specificity and any nonspecific amplification.

### Hemolysis Calculation

Samples that have undergone hemolysis may show artificially increased miRNA signals, which could distort subsequent results. The gold-standard method for assessing hemolysis is considered a comparison of two miRNA, where miR-23a-3p is stable and not affected by hemolysis while the expression of miR-451a is erythrocyte specific with increased concentrations when hemolysis is present, as described in, for example, Smith M D, et al., “Haemolysis detection in microRNA-seq from clinical plasma samples,” Genes, 13 (7): 1288 (2022). A delta Cq (ΔCq) for miR-23a-3p minus miR-451a was therefore calculated, where larger values indicate potential hemolysis, as described in, for example, Blondal T, et al, “Assessing sample and miRNA profile quality in serum and plasma or other biofluids,” Methods, 59 (1): S1-6 (2013). Those samples having ΔCq≥7 were selected for further in-depth evaluation according to standard protocols (see Smith (2022) above). The highest result was 8.66 (S141 from January 2023) with a handful of samples returning values ≥7≤8.30. The miRNA Cq values for these samples were otherwise within acceptable ranges as determined using the interquartile range (Q1=30.6, Q3=34.9, IQR=4.3, interquartile multiplier value k=1.5), so they were not excluded from further analysis.

### Relative Quantitation Using 2^{-(ΔΔCq)}

A NormFinder analysis confirmed the stability of miRNAs used for normalization and the same miRNA as those used in prior experiments were selected for consistency and reproducibility (miR-146a-5p, miR-29b-3p, and miR-126-5p). The geometric mean was used for relative quantitation/normalization and a suitability check following the protocol of Vandesompele was performed (Vandesompele J, et al., “Accurate normalization of real-time quantitative RT-PCR data by geometric averaging of multiple internal control genes,” Genome Biol., 3(7): 1-12 (2002)). Since the standard deviation of the ratio V ¾ was less than 0.15, no additional reference miRNAs were needed. Gene fold expression changes were calculated using 2^{-(ΔΔCq)}, with ΔΔCq calculated as the normalized sample Cq value minus the mean of the normalized control sample Cqs. Fold regulation was calculated as relative mean gene fold expression changes in ALS/controls, Parkinson's disease/ALS, and primary lateral sclerosis/ALS, respectively, and fold regulation was defined as equal to gene fold change when greater than one and as negative one divided by gene fold change when fold change was less than one.

### Statistical Analyses

The skewed distribution of the data which varied between miRNA suggested that a non-parametric statistical analyses would be preferable. A Kruskal-Wallis test was performed to assess two alternative hypotheses at the level of p<0.05:
H₀: The gene fold changes, 2^{-(ΔΔCq)}, of miRNA from all disease groups were the same.
H₁: The gene fold changes, 2^{-(ΔΔCq)}, of miRNA from all disease groups were not the same.
   If the null hypothesis H₀ were to be rejected, we would perform post-hoc analyses on gene fold change values (2^{-(ΔΔCq)}) between the following pairs of disease groups, ALS versus Controls, ALS versus primary lateral sclerosis, ALS versus Parkinson's disease, using Mann Whitney tests, at the level of p<0.05 to test the following alternative hypotheses:
H₀: There is no difference in the median gene fold change, 2^{-(ΔΔCq)}, for miRNA between the two disease groups.
H₁: There is a difference in the median gene fold change, 2^{-(ΔΔCq)}, for miRNA between the two disease groups.

Individual miRNA (2^{-(ΔΔCq)}) representing values greater than four standard deviations from the mean were identified as extreme outliers and removed before gene fold expression calculation (one from Parkinson's disease and five from control samples). Three samples were considered invalid and removed entirely from gene fold expression calculations (1 ALS, 1 Control, 1 primary lateral sclerosis) because ≥50% of the miRNA values exceeded four standard deviations from the mean suggesting that the plasma samples from these individuals was compromised. Although nothing was flagged for these samples in the quality control measures built into the methods described herein, plasma samples were collected, processed, and stored initially by numerous individuals across the USA, and consequently, finding three samples of concern out of 331 total represents less than one percent of the total samples analyzed.

Sensitivity and specificity were evaluated using normalized ΔCq values. Sensitivity is a measure of the ability of a screening test to positively classify someone who has the disease. It is calculated as a percent identification of true positive cases in the dataset divided by the total number of individuals with the disease, as defined by a gold standard method of disease identification, described in, for example, Trevethan R, “Sensitivity, Specificity, and Predictive Values: Foundations, Pliabilities, and Pitfalls in Research and Practice,” Front Public Health, 5:30 (2017). The current gold standard for an ALS diagnosis is a clinical evaluation by a neurologist who has evidence of progressive upper and lower motor neuron degeneration as seen in EMG, the ALSFRS-R score, and forced vital capacity (FVC) based upon ratified consensus criteria (Verma A, “Clinical Manifestation and Management of Amyotrophic Lateral Sclerosis in Amyotrophic Lateral Sclerosis. In: Toshiyuki A, ed.,” Amyotrophic Lateral Sclerosis, Exon Publication; 1-14 (2021)). Specificity is a measure of the ability of a screening test to accurately classify someone who does not have the disease, comparing true negative cases to the total number of individuals without the disease in the dataset. Also important for biomarker evaluation is a measure of positive predictive value (PPV) for the test, which compares true positive cases with false positive identification. The negative predictive value (NPV) of a test is the number of true negative cases identified by the test relative to false negative identification (see Trevethan above).

Three orthogonal measures were used to determine ALS classification accuracy against healthy controls in Rstudio. First, a logistic regression was calculated to determine a Receiver Operator Characteristics curve (ROC) using only the current data and a random 80% split for training and 20% for testing purposes. Values from all ALS and non-neurological controls (n=269) were included in the model without removing outliers. Seed set was 375 and all miRNA data were included in this model. Second, a random forest machine learning algorithm was used applying all of the current data from this paper (n=269) without removing outliers and a 79% split between training and testing data. The model was tested with a seed set of 375 and iteratively removing miRNA with low model contribution. Third, a second random forest algorithm was used testing the current data set (n=269) against a training set comprised of ΔCq values from an independent cohort previously published (n=100, Banack et al. (2020) Table 1, below). The model was tested with a seed set of 101 and by iteratively removing miRNA with low contributions to the classification. Parkinson's disease and primary lateral sclerosis samples were not included in these models due to sample sizes that were too low to give meaningful results. Hanczar, B, Hua, et al., “Small-sample precision of ROC-related estimates,” Bioinformatics, 26 (6): 822-830 (2010).

TABLE 1

| | | | | | Data from Banack et | |
|---|---|---|---|---|---|---|
| | | | | | al. 2022 used for | |
| | | | | | Machine Learning | |
| | Amyotrophic | | | *Primary | Training Data Set | |
| | Lateral | Healthy | Parkinson's | Lateral | | Healthy |
| | Sclerosis | Control | Disease | Sclerosis | ALS | Control |
| Total Samples | 119 | 150 | 20 | 42 | 50 | 50 |
| Male/Female | 72/28 | 110/40 | 14/6 | 24/17 | 35/15 | 30/20 |
| Age Group | | | | | | |
| <30 | 0 | 5 | 0 | 0 | 2 | 14 |
| 30-39 | 3 | 17 | 0 | 1 | | 13 |
| 40-49 | 14 | 15 | 0 | 5 | 7 | 9 |
| 50-59 | 21 | 86 | 5 | 12 | 19 | 10 |
| 60-69 | 32 | 26 | 3 | 16 | 15 | 4 |
| 70-79 | 28 | 1 | 9 | 4 | 6 | 0 |
| 80-89 | 2 | 0 | 3 | 3 | 1 | 0 |
| ALSFRS-R | | | | | | |
| Mean, Median (Min-Max) | 32, 33.5 | | | | | |
| | (6-45) | | | | | |
| Early stage ≥35 ALSFRS | 50 | | | | | |
| Late stage <35 ALSFRS | 50 | | | | | |
| Progression | | | | | | |
| ALSFRS-R slope <−0.5 | 9 | | | | 8 | |
| ALSFRS-R slope −0.5 to −1.0 | 9 | | | | 10 | |
| ALSFRS-R slope >−1.0 | 0 | | | | 6 | |
| Onset | | | | | | |
| Speech and/or swallowing muscles | 8 | | | | 5 | |
| Arm or Hand | 25 | | | | 8 | |
| Neck, Back, or Abdominal area | 7 | | | | 0 | |
| Leg or Foot | 35 | | | | 8 | |
| Breathing Muscles | 2 | | | | | |
| All over body | 2 | | | | | |
| ALS recognized in other family | 3 | | | | 0 | |
| members | | | | | | |
| Months since diagnosis | | | | | | |
| Mean, Median (Min-Max) | 16.5, 6.5 | | 59, 54.5 | 68.1, 51.9 | | |
| | (2-122) | | (2-178) | (9.3-172.9) | | |
| Other | | | | | | |
| Idiopathic Parkinson's Disease | | | 13 | | | |
| Parkinson-Plus Syndromes | | | 7 | | | |

Table 1 represents Characteristics of study cohort. * Primary Lateral Sclerosis samples from the natural history study (Mitsumoto et al. 2023). +Disease duration at baseline. Amyotrophic Lateral Sclerosis (ALS) progression data were calculated on all samples wherein at least two slopes were provided in the accompanying database. Complete individual characteristics for all patients were not available in the respective databases.

### Results

A comparison of the eight-miRNA ALS fingerprint between 119 ALS and 150 healthy controls indicated identical fold regulation direction with similar magnitude to those found in prior published studies (Table 2 below). A comparison of median gene fold change between ALS, Parkinson's disease, primary lateral sclerosis and healthy controls using a Kruskal-Wallis analysis was highly significant for all eight-miRNA with H values ranging from 38 to 136 (critical value=16, p<0.001), suggesting that at least one of the disease groups was not from the same population. Table 2 below shows the fold regulation of an 8 miRNA ALS fingerprint across 449 independent plasma samples over 4 unique cohort experiments, and reveals remarkable consistency in both direction and values. Negative numbers (in parenthesis) indicated down-regulation of miRNA in ALS relative to controls and positive numbers (in bold) indicated up-regulation. Prior published fold regulation data are from Banack (2020) and Banack (2022).

### Table 2

Table 2. Fold regulation of an eight miRNA ALS fingerprint across 449 independent plasma samples over four unique cohort experiments showed remarkable consistency in both direction and values. Negative numbers (in parenthesis) indicated down-regulation of miRNA in ALS relative to controls and positive numbers indicate up-regulation.

| | Fold Regulation | | | |
|---|---|---|---|---|
| | Current | Banack et al.²³ | Banack et al.²² | Banack et al.²² |
| | (n = 269) | (n = 100) | Experiment 1 (n = 40) | Experiment 2 (n = 40) |
| miR-10b-5p | (−7.38) | (−3.2) | (−2.1) | (−7.0) |
| miR-4454 | (−2.55) | (−2.7) | (−1.7) | (−1.8) |
| miR-199a-3p | 2.03 | 1.0 | 1.4 | 2.7 |
| miR-151a-3p | 2.09 | 1.1 | 1.5 | 2.2 |
| miR-151a-5p | 1.77 | 1.1 | 1.4 | 3.2 |
| miR-199a-5p | 2.54 | 1.1 | 1.9 | 4.2 |
| miR-146a-5p | 1.57 | 1.1 | 1.2 | 1.4 |
| miR-29b-3p | (−1.35) | (−1.5) | (−1.7) | (−1.7) |

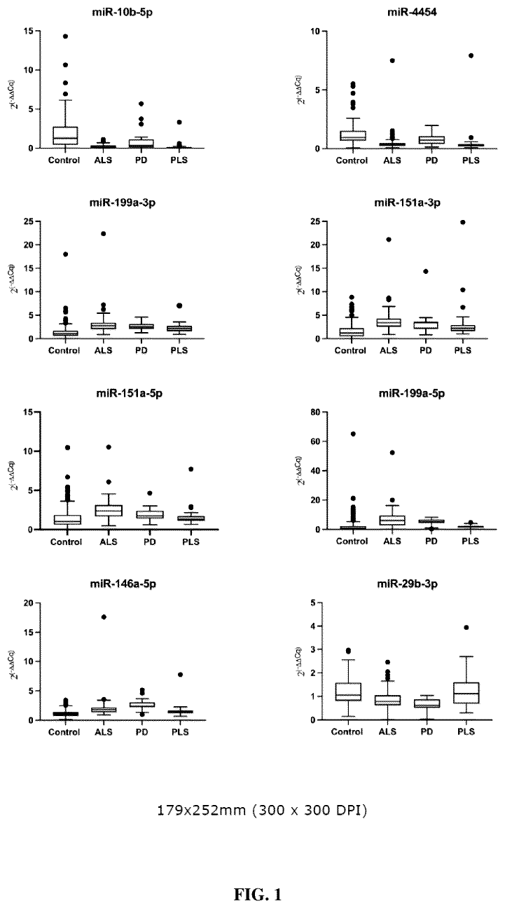

Given the non-normal distribution of miRNA fold change values (FIG. 1), a post-hoc Mann Whitney analyses was used to compare the median gene fold change (2^{-(ΔΔCq)}) for each miRNA for the following disease groups: ALS versus healthy controls, Parkinson's disease versus ALS, and primary lateral sclerosis versus ALS. For all eight-miRNAs, the gene fold change between ALS and control samples had p-values <0.05 (Table 3). The same was true between primary lateral sclerosis and ALS. For the comparison between Parkinson's disease and ALS, five out of eight-miRNAs were different. Interestingly, the three miRNA that were not identified as different between ALS and Parkinson's disease on the Mann Whitney analysis were also not different in another study comparing ALS with controls and may be an artifact of a small sample size. Table 3 provides the fold regulation of ALS miRNA fingerprints, as determined by qPCR from LICAM enriched EV extractions of blood plasma. Z-statistic was from a two-tailed Mann Whitney post-hoc analysis. Median values are reported as gene fold expression (2^{-(ΔΔCq)}. Z-statistic are reported as absolute values.

### Table 3

Table 3. Fold regulation of ALS miRNA fingerprint, as determined by qPCR from LICAM enriched EV extractions of blood plasma. Z-statistic was from a two-tailed Mann Whitney post-hoc analysis. Median values are reported as gene fold expression (2^{(-ΔΔCq)}. Z-statistic are reported as absolute values.

Amyotrophic Lateral Sclerosis (ALS) versus Controls

| | | | Median | Median | | |
|---|---|---|---|---|---|---|
| | | | ALS | Control | Fold | |
| | p-value | Z-statistic | (n = 119) | (n = 150) | Regulation | Regulation |
| miR-10b-5p | p < 0.00001 | 10.39 | 0.15 | 1.27 | (−7.38) | down- |
| | | | | | | regulated |
| miR-4454 | p < 0.00001 | 9.97 | 0.38 | 0.95 | (−2.55) | down- |
| | | | | | | regulated |
| miR-199a-3p | p < 0.00001 | 9.76 | 2.74 | 1.07 | 2.03 | up-regulated |
| miR-151a-3p | p < 0.00001 | 9.49 | 3.39 | 1.18 | 2.09 | up-regulated |
| miR-151a-5p | p < 0.00001 | 8.46 | 2.37 | 1.02 | 1.77 | up-regulated |
| miR-199a-5p | p < 0.00001 | 9.02 | 6.00 | 0.93 | 2.54 | up-regulated |
| miR-146a-5p | p < 0.00001 | 8.66 | 1.81 | 1.04 | 1.57 | up-regulated |
| miR-29b-3p | p < 0.00001 | 4.87 | 0.78 | 1.05 | (−1.35) | down- |
| | | | | | | regulated |

Parkinson's Disease (PD) versus ALS

| | | | Median PD | Median ALS | Fold | |
|---|---|---|---|---|---|---|
| | p-value | Z-statistic | (n = 20) | (n = 119) | Regulation | Regulation |
| miR-10b-5p | p < 0.05 | 2.35 | 0.36 | 0.15 | 4.21 | up-regulated |
| miR-4454 | p < 0.001 | 3.58 | 0.72 | 0.38 | 1.64 | up-regulated |
| miR-199a-3p | NS | 0.71 | 2.54 | 2.74 | (−1.06) | down- |
| | | | | | | regulated |
| miR-151a-3p | NS | 0.95 | 3.34 | 3.39 | (−1.20) | down- |
| | | | | | | regulated |
| miR-151a-5p | p < 0.05 | 2.55 | 1.74 | 2.37 | (−1.27) | down- |
| | | | | | | regulated |
| miR-199a-5p | NS | 1.08 | 5.31 | 6.00 | (−1.23) | down- |
| | | | | | | regulated |
| miR-146a-5p | p < 0.00001 | 4.11 | 2.37 | 1.81 | 1.46 | up-regulated |
| miR-29b-3p | p < 0.05 | 2.46 | 0.62 | 0.78 | (−1.37) | down- |
| | | | | | | regulated |

Primary Lateral Sclerosis (PLS) versus ALS

| | | | Median PLS | Median ALS | Fold | |
|---|---|---|---|---|---|---|
| | p-value | Z-statistic | (n = 42) | (n = 119) | Regulation | Regulation |
| miR-10b-5p | p < 0.05 | 2.41 | 0.08 | 0.15 | (−1.80) | down- |
| | | | | | | regulated |
| miR-4454 | p < 0.05 | 2.32 | 0.27 | 0.38 | (−1.54) | down- |
| | | | | | | regulated |
| miR-199a-3p | p < 0.001 | 3.32 | 2.13 | 2.74 | (−1.32) | down- |
| | | | | | | regulated |
| miR-151a-3p | p < 0.00001 | 4.30 | 2.22 | 3.39 | (−1.37) | down- |
| | | | | | | regulated |
| miR-151a-5p | p < 0.05 | 2.32 | 0.27 | 0.38 | (−1.54) | down- |
| | | | | | | regulated |
| miR-199a-5p | p < 0.00001 | 6.67 | 1.79 | 6.00 | (−3.24) | down- |
| | | | | | | regulated |
| miR-146a-5p | p < 0.001 | 3.63 | 1.50 | 1.81 | (−1.29) | down- |
| | | | | | | regulated |
| miR-29b-3p | p < 0.01 | 2.98 | 1.12 | 0.78 | 1.36 | up-regulated |

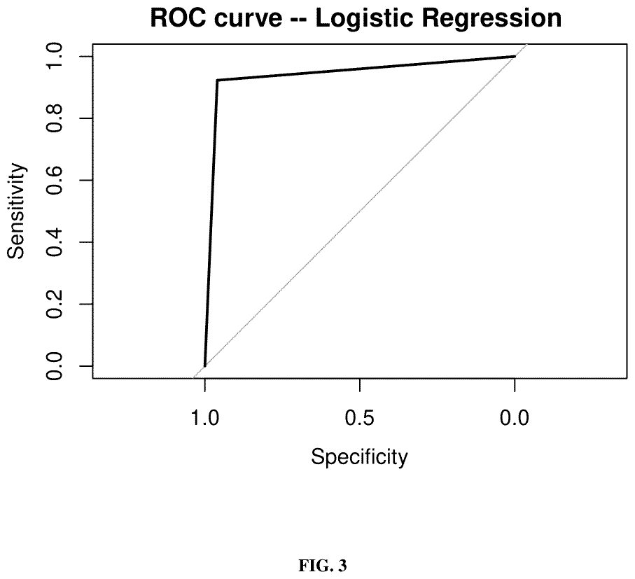

Classification accuracy was tested using three orthogonal measures (Tables 4,5,6). Using only the current data of ALS and healthy controls (n=269) and a logistic regression model (Table 5) with a random data split, sensitivity and negative predictive value were both at 100% while specificity and positive predictive value were at 97% and 96% respectively (Table 6). A receiver operator characteristics curve demonstrated an area under the curve of 98% (FIG. 3). Deviance residuals for the model were as follows: Min=−2.10, 1^{rst} Quartile=−0.28, Median=0.03, 3^{rd} Quartile=0.24, Max=3.79, null deviance=296.8 (215 degrees of freedom), residual deviance=96.3 (207 degrees of freedom). Akaike information criterion was 114.3, f1 score was 98%, and the number of Fisher Scoring iterations was 7. A random forest analysis with a random split, also using only the current data, had good sensitivity, specificity, positive predictive value, and negative predictive value all between 96-97% (Table 6). Good results in this analysis used only six of the eight-miRNA (mi-29b-3p and miR-146a-5p were removed) and the contribution of each remaining miRNA was determined (Table 4). When the same data were tested against a training data set pulled from an independent cohort of samples (Banack (2022)), sensitivity dropped to 82% but specificity remained at 97% (Table 6).

Table 4 provides a random forest machine learning analysis, where Mean Decrease in Gini is a measure of the contribution of each miRNA to the homogeneity of the random forest trees where an increased value represents a larger contribution to classification criteria. Random Split data were exclusively from the current data set with a 79% random split. Separate Cohorts used the current data set to test against the training data published in Banack (2022) with used plasma from an independent sample cohort.

### Table 4

Table 4: Random forest machine learning analysis. Mean Decrease in Gini is a measure of the contribution of each miRNA to the homogeneity of the random forest trees where an increased value represents a larger contribution to classification criteria. Random Split data are exclusively from the current data set with a 79% random split. Separate Cohorts used the current data set to test against the training data, previously published²³, which used plasma from an independent sample cohort

Mean Decrease Gini

| | | Separate Cohorts: |
|---|---|---|
| | Random Split | Train²³ n = 100; Test |
| | (n = 269) | n = 269 |
| miR-10b-5p | 33.6 | 9.0 |
| miR-4454 | 21.4 | 9.6 |
| miR-199a-3p | 17.4 | 12.2 |
| miR-151a-3p | 15.8 | 4.9 |
| miR-151a-5p | 9.1 | 7.7 |
| miR-199a-5p | 7.8 | 6.0 |

Table 5 below provides the logistic regression model used to calculate the receiver operator characteristics (ROC) curve shown in FIG. 3.

TABLE 5

| | Estimate Std | Std. Error | z value | Pr(>\|z\|) |
|---|---|---|---|---|
| Intercept | −4.58 | 1.76 | −2.61 | 0.009** |
| miR-10b-5p | −0.21 | 0.17 | −1.26 | 0.21 |
| miR-4454 | −1.15 | 0.29 | −3.98 | 6.84e−05*** |
| miR-199a-3p | 1.22 | 0.50 | 2.43 | 0.01* |
| miR-151a-3p | 0.48 | 0.30 | 1.60 | 0.11 |
| miR-151a-5p | 1.17 | 0.37 | 3.19 | 0.001** |
| miR-199a-5p | −0.11 | 0.23 | −0.47 | 0.64 |
| miR-146a-5p | 1.59 | 0.62 | 2.55 | 0.011* |
| miR-29b-3p | 1.72 | 0.46 | 3.70 | 0.0002*** |

***p < 0.001,
**= p < 0.01,
*= p < 0.05

Table 6 below provides results from the three orthogonal models that were used to interrogate ALS classification between ALS and healthy controls.

TABLE 6

| | | | Positive | Negative | Overall | | | Data |
|---|---|---|---|---|---|---|---|---|
| | Sensitivity | Specificity | Predictive | Predictive | Accuracy | # miRNA | Sample | Source |
| | (%) | (%) | Value (%) | Value (%) | (%) | included | Size | (details) |
| 1. Random | 96 | 97 | 96 | 97 | 96 | 6 | n = 269 | Current |
| forest split | | | | | | | | data (79% |
| | | | | | | | | split) |
| 2. Random | 82 | 97 | 95 | 87 | 90 | 6 | n = 369 | Current |
| forest separate | | | | | | | | data (Test, |
| cohorts | | | | | | | | n = 269) + |
| | | | | | | | | Train |
| | | | | | | | | data²³ |
| | | | | | | | | (n = 100) |
| 3. Logistic | 100 | 97 | 96 | 100 | 98 | 8 | n = 269 | Current |
| Regression | | | | | | | | data (80% |
| | | | | | | | | split) |

### Example 2—Using the miRNA Fingerprint of miRNA for Amyotrophic Lateral Sclerosis/Motor Neuron Disease without Isolating Neural-Derived Exosomes

Using retrospective blood samples from previously identified ALS and healthy controls, the eight-miRNA fingerprint of Example 1 was evaluated in circulating blood, without exosome extraction, as an aid to diagnosing ALS.

Plasma samples were collected in EDTA tubes from a total of 211 ALS patients and 196 control samples and were separated into three cohort groups (Table 7). The majority of ALS samples were obtained from the USA National ALS Biorepository maintained by the Centers for Disease Control and Prevention, and the Agency for Toxic Substances and Disease Registry (CDC, Advarra IRB Pro00053269) with additional ALS blood samples obtained through a Phase Ila clinical trial (NCT03580616, Dartmouth Institutional Review Board D18095, collected between 2019-2022). Control samples were obtained from Innovative Research Inc (Novi, MI, FDA Approval, #3003372368, collected across the USA prior to 2022) and Precision for Medicine (Norton, MA, CR00425931, collected across the USA from 2020-2022) and were all reported to be healthy controls. All blood samples were collected in K₂EDTA tubes and samples blinded before being processed and analyzed. Every patient sample was from a unique individual with no overlap between cohorts.

TABLE 7

| | | | | NEE Fold Regulation | | | |
|---|---|---|---|---|---|---|---|
| | | | | Banack et | Banack et | | |
| | | | | al. 2020 | al. 2020 | | |
| | Circulating Fold Reg | | | Experiment | Experiment | Banack et | Banack et |
| | Cohort 1 | Cohort 2 | Cohort 3 | 1 | 2 | al. 2022 | al. 2024 |
| 151a-3p | 3.5 | 2.0 | 3.1 | 1.5 | 2.2 | 1.1 | 2.1 |
| 151a-5p | 2.3 | 2.3 | 2.4 | 1.4 | 3.2 | 1.1 | 1.8 |
| 146a-5p | 2.3 | 1.6 | 2.6 | 1.2 | 1.4 | 1.1 | 1.6 |
| 4454 | (−10.3) | (−10.9) | (−10.7) | (−1.7) | (−1.8) | (−2.7) | (−2.6) |
| 10b-5p | (−23.0) | (−13.1) | (−17.0) | (−2.1) | (−7.0) | (−3.2) | (−7.4) |
| 199a-3p | 2.7 | 2.3 | 3.6 | 1.4 | 2.7 | 1 | 2.0 |
| 199a-5p | 3.8 | 3.4 | 6.7 | 1.9 | 4.2 | 1.1 | 2.5 |
| 29b-3p | (−1.3) | (−1.3) | (−1.6) | (−1.7) | (−1.7) | (−1.5) | (−1.4) |
| n = ALS | 20 | 132 | 59 | 20 | 20 | 50 | 119 |
| n = Controls | 20 | 117 | 59 | 20 | 20 | 50 | 150 |

Table 7 shows the fold regulation for eight ALS-related miRNAs comparing values determined by analysing circulating blood plasma without exosome isolation (“Circulating Fold Reg”), to values determined by the previous method of analysing isolated neural-enriched extracellular vesicles (“NEE Fold Regulation). The direction of fold-regulation remained the same between groups, but the magnitude of the fold changes were increased in miR-4454 and miR-10b-5p in the circulating miRNA.

The data from “Cohort 1” were analyzed from blood plasma miRNA representing 20 ALS patients and 20 controls. The data from “Cohort 2” included 132 ALS patient plasma and 117 controls. “Cohort 3” came from 59 ALS patients and 59 controls.

### RNA Extraction

In order to compare the circulating miRNA results with previous data obtained from isolated exosomes (“NEE”), the same plasma samples of Example 2 extracted total RNA without first isolating NEE. A slightly different protocol for total RNA extraction from plasma which varies by the volume of lysis reagent and number and volume of ethanol washes was used.

Control and ALS plasma was removed from an −80° C. freezer, thawed at room temperature, then 200 μL aliquoted into a 1.5 mL LoBind® Tube (Eppendorf). Total RNA was extracted using the Qiagen miRNeasy Serum/Plasma Kit (50) Cat. No./ID: 217184 according to the manufacturer's instructions. Briefly, 1 mL Qiazol Lysis buffer, containing 1 μL UniSp2, 4 and 5 spike-in, was added to each sample and mixed by pipetting up and down several times. Samples were incubated at RT for 5 mins. An equal volume of chloroform (200 μL) was added to each sample, then the tubes were vigorously shaken for 15 seconds. The samples were incubated at RT for 3 mins, then centrifuged at 12,000×g for 15 min at 4° C., to generate three distinct layers. The top aqueous layer containing RNA was carefully removed without touching the middle layer containing DNA and placed in a new clean 2 mL LoBind® tube. To this were added 1.5 volumes of 100% ethanol (to 700 μL RNA was added 1050 μL 100% ethanol). The samples were mixed thoroughly by pipetting up and down. To a spin column, 700 μL sample was added, then centrifuged at 10,000×g for 15 secs at RT, then the eluent was discarded. This step was repeated until all the sample had been added to the spin column (three spins).

About 700 μL Buffer RWT then was pipetted into each column, then spun at 10,000×g for 15 secs at RT. The eluent was discarded. Next, about 500 μL Buffer RPE was added to the spin-column then centrifuged at 10,000×g for 15 secs at RT, and the eluent was discarded. About 500 μL 80% ethanol then was added to the spin column then spun for 2 min at 10,000×g at RT, and the eluent discarded. The spin-column was placed in a new 2 mL collection tube (with no lid), the lid opened, and centrifuged at 17,000×g for 5 min, RT to dry the spin column. Finally, the spin column was placed in a clean 1.5 mL LoBind® tube, and 15 μL nuclease-free water added directly to the membrane. The spin-columns were incubated at RT for 1 min, then spun for 1 min at 17,000×g to elute the RNA. The RNA was either immediately used to make cDNA, or frozen at −80° C. until required.

### CDNA Synthesis

cDNA was synthesised as described previously (Dunlop et al 2021), and according to the manufacturer's instructions using the Qiagen miRCURY LNA RT Kit (#339340). cDNA was synthesised in duplicate, and to each 10 μL reaction were added 0.5 μL of UniSp6 and cel-miR-39-3p spike-ins (UniSp6 is a part of the miRCURY LNA RT Kit #339340 and cel-miR-39-3p is part of the miRCURY LNA RNA Spike-in Kit #339390. For instructions on how to reconstitute these, see RNA Spike-in Kit, for RT, Handbook #HB-2433-002). About 4 μL total RNA was used in the cDNA synthesis reaction, since it was previously demonstrated that 4 μL returns a more robust Cq in genes of low copy number (Dunlop et al., 2021). The conditions for the RT reaction were as follows: 95° C. 5 min, 42° C. 60 mins, 4° C. HOLD. Duplicate cDNA reactions were pooled into a final volume of 20 μl, then aliquoted into 3 μL aliquots before being stored at −20° C.

For each sample, quality control was conducted prior to downstream qPCR analysis. Each sample was assayed for the following spike-ins; UniSp2, 4 and 5 to measure the effectiveness of the RNA extraction step, and UniSp6 and cel-miR-39-3p to measure the success of the cDNA/RT reaction step. All samples were deemed to have passed QC if the Cqs for spike-ins varied by less than two standard deviations from the mean raw Cq for each target.

### Sample Signal

As a method for determining whether there was sufficient cDNA in each sample to proceed with downstream reactions, four miRNAs were measured that were collectively referred to as “sample signal.” Four miRNA that were predicted to be either in low or high copy-number in human plasma were measured; miR-142-3p, miR-451a, miR-23a-3p and miR-103a-3p. The presence of these targets with raw Cqs less than 34 constituted a “pass” for use in downstream experiments.

### Hemolysis Calculation

Certain miRNAs are enriched in red blood cells, and if hemolysis occurs during plasma processing, the release of these miRNAs into the plasma can artefactually affect results. Thus, miR-451a, miR-23a-3p were measured, and delta Cq for miR-23a-3p-miR-451a was calculated. Any delta Cq that was ≥7 indicated hemolysis may have occurred and these samples should be assessed.

### Efficiency Calculations

The efficiency of primer pairs to amplify the target was calculated in two ways: Using the standard curve method, and the LinRegPCR method. For the standard curve method, cDNA was randomly pooled from the total sample set. A cDNA dilution series of six samples diluted ½ was prepared from an initial dilution of ⅕ into nuclease-free water. Standard curves were run in triplicate, and data acquired in Maestro version 2,3 on the BioRad Opus CFX 384. Reaction conditions were as follows: initial activation, 2 min, 95° C.; 2-step cycling: denaturation, 10 secs, 95° C.; combined annealing/extension; 60 secs, 56° C.; for a total of 40 cycles. Efficiency was calculated by Maestro 2.3 from the slope of each standard curve. Primer efficiencies between 94% and 102.7% were achieved. Individual reaction efficiencies for every sample were also calculated in LinRegPCR using the slope determined from the window-of-linearity for each individual reaction. An average PCR efficiency was calculated for all samples in an amplicon group, and the average efficiencies for all primer pairs should be between 1.80 and 2.00.

### RNA Extraction Efficiency

Prior to lysing plasma samples, 1 μL of combined UniSp2, Unip4 and UniSp5 synthetic spike-in was added to each 1 mL of Qiagen QIAzol® Lysis Reagent (#79306, Lot No. 57204842). Spike-ins were prepared according to the manufacturer's instructions (Qiagen miRCURY LNA RNA Spike-in Kit #339390, Lot No. PA03). RNA extraction efficiency was determined by measuring UniSp2, 4 and 5 in all samples by qPCR using miRCURY LNA miRNA PCR assays #339306.

Real-Time PCR (qPCR) and Analysis

cDNA was removed from the freezer (−20° C.) and diluted 1/30 into nuclease-free water. qPCR was conducted using Qiagen miRCURY LNA PCR Assays #339306 according to the manufacturer's instructions. Preparation of 384-well PCR plates was conducted on the Opentrons OT-2 liquid-handling robot using a custom protocol designed in Python 3.0. To 7 μL master mix, 3 μL sample was added to each well and mixed five times using the robot. Each plate contained three no-template controls per gene and triplicate inter-plate calibrators to allow for cross-plate comparison (TATAA, Sweden). qPCR was run on the BioRad CFX Opus 384 using the following conditions: initial activation, 2 min, 95° C.; 2-step cycling: denaturation, 10 secs, 95° C.; combined annealing/extension; 60 secs, 56° C.; for a total of 40 cycles. A melt-curve (60-95° C.) for each primer was used to check for non-specific amplification using the sample maximization method. Cq accounting for efficiency was determined from the double derivative method using LinRegPCR.

Cq values were normalized using the geometric mean of three stable reference genes (miR-146a-5p, miR-29b-3p, and miR-126-5p) and a suitability test was calculated to assess suitability (Banack et al. 2024, Vandesompele et al. 2002). Gene-fold expression, 2^{-(AACq)}, was calculated from delta-delta Cq (normalized sample Cq minus the mean of normalized control sample Cqs). Fold regulation was calculated using the mean gene fold expression for ALS divided by the mean gene fold expression for control samples. For this analyses values greater than four standard deviations were considered extreme outliers and were eliminated from the analysis for that specific miRNA alone (n=0 for circulating cohort 1; n=6 for circulating cohort 2; and n=8 for circulating cohort 3). Fold regulation was defined as equal to gene fold change when it was greater than one and was calculated as negative one divided by gene fold change when the fold change was less than one.

### Digital PCR

Total RNA was extracted from 200 μL plasma samples and cDNA synthesised as normal. Twenty-four of these RNA samples were simultaneously synthesised in triplicate into cDNA, and from the extra reaction, 2 μL was aliquoted and diluted with 58 μL water to make a dilution of 1/30. Diluted samples were frozen at −20° C. until needed. For the dPCR reaction, an 8500 partition 96-well plate was used on the Qiagen QIAcuity instrument. In each 15 μL dPCR reaction, 3 μL of diluted cDNA and 1.5 μL primer was used ( 1/10 dilution, as per the qPCR reaction). To allow for direct comparison, the exact same primers were used for this experiment at the same dilution ( 1/10) as qPCR comparisons. dPCR and qPCR data were normalized, to account for variations across samples in RNA concentrations, using primers 146a-5p, 29b-3p, and 126-5p. The normalization was performed using Equations 1 through 4. dPCR data was not obtained for all samples with primer 29b-3p; those samples without a measured copies per microliter were assigned a copies per microliter value of 0.25 thus facilitating the calculation of a geometric mean.

$$( qPCR data normalization ) Δ {C}_{qprimeri , samplej} = {C}_{qprimeri , samplej} - {C}_{qgeometricmean , samplej} \qquad Eq . 1$$

$$( dPCR data normalization ) {β}_{primeri , samplej} = \frac{{α}_{primeri , samplej}}{{α}_{geometricmean , samplej}} \qquad Eq . 2$$

$${C}_{qgeometricmean , samplej} = {( {C}_{q 146 a - 5 p , samplej} \times {C}_{q 29 b - 3 p , samplej} \times {C}_{q 126 - 5 p , samplej} )}^{1 / 3} \qquad Eq . 3$$

$${α}_{geometricmean , samplej} = {( {α}_{146 a - 5 p , sample} \times {α}_{29 b - 3 p , samplej} \times {α}_{126 - 5 p , samplej} )}^{1 / 3} \qquad Eq . 4$$

where: ΔCq is the concentration normalized Cq
   α is the measured copies per microliter
   β is the concentration normalized copies per microliter
Fold regulation was calculated for qPCR and dPCR using Equations 5 through 9.

$$Δ Δ {C}_{qprimeri , samplej} = Δ {C}_{qprimeri , samplej} - median Δ {C}_{qprimeri , forallcontrolsamples} \qquad Eq . 5$$

$$Foldregulation ( qPCR ) = medianALS {2}^{- ΔΔ {C}_{qprimeri}} / mediancontrol {2}^{- ΔΔ {C}_{qprimeri}} \qquad Eq . 6$$

$${Δβ}_{primeri , samplej} = {β}_{primeri , samplej} - median {β}_{primeri , forallcontrolsamples} \qquad Eq . 7$$

$$Foldregulation ( dPCR ) = medianALS {2}^{- {Δβ}_{primeri}} / mediancontrol {2}^{- {Δβ}_{primeri}} \qquad Eq . 8$$

$$when Foldregulation ( qPCR ⋁ dPCR ) < 1 Foldregulation = 1 / - Foldregulation \qquad Eq . 9$$

A comparison between qPCR and dPCR was made for eight miRNAs, and is shown in FIG. 3. As shown in the Figure, all miRNAs that were previously statistically significant and differentiated ALS from controls were also highly significant when quantitated using dPCR. All samples were normalized to a geometric mean of 146a-5p, 29b-3p, 126-5p. Unpaired non-parametric T-test, Mann-Whitney post-hoc analysis in GraphPad Prism version 10 was used.

Fold-regulation direction was the same in dPCR, but the change was of smaller magnitude in upregulated genes and larger in down-regulated genes, as shown in Table 8 below. The fold regulation in Table 8 was defined as equal to fold change (median ALS/median Control) when greater than one, and as negative one divided by fold change when fold change was less than one. The direction of fold-regulation was mostly reproduced for dPCR, but the magnitude was smaller for positive changes, and larger for negative changes.

TABLE 8

| | 151a-3p | 151a-5p | 146a-5p | 4454 | 10b-5p | 29b-3p | 199a-3p | 199a-5p | 107 | 150-5p | let-7g-5p | 126-5p |
|---|---|---|---|---|---|---|---|---|---|---|---|---|
| Fold | 1.88 | 1.07 | 1.27 | −8.84 | −5.84 | −1.01 | 2.50 | 1.80 | −1.18 | −13.43 | −3.65 | −2.63 |
| regulation | | | | | | | | | | | | |
| dPCR | | | | | | | | | | | | |
| Fold | 2.92 | 1.14 | 3.21 | −7.8 | −10.55 | −1.55 | 4.04 | 6.04 | −1.43 | −7.46 | −2.82 | −1.9 |
| regulation | | | | | | | | | | | | |
| qPCR | | | | | | | | | | | | |

### Classification Algorithms

Data were split into two datasets in order to create a prediction model for the assessment of accuracy, sensitivity, and specificity. miRNA from the first 274 patients was used as the training data. The miRNA from the last 118 patients was used as the test data. The choice of training and testing data came by combining cohorts with the first two cohorts combined to create the training set and the third cohort used to test accuracy. Each data point represented a unique patient without duplication. Because there were 15 more ALS patients in the first two cohorts than controls and we desired an equal division between ALS and controls for the training set, the last 15 ALS patients run were included in the test data set. All eight miRNA identified as our ALS fingerprint were considered for inclusion in the model and removal of individual miRNA were conducted with iterative model testing. Outliers were not removed from analyses. Two models, random forest and a general logistic model, were created and compared using R-studio.

Using two independent data sets with no overlap in patient samples, two classification models with predictive accuracies of about 97% were created, as shown in Table 9 below. The models were the Random Forest and Logistic Regression classification models.

TABLE 9

| | Random Forest (%) | Logistic Regression (%) |
|---|---|---|
| Accuracy | 97 | 97 |
| Sensitivity | 95 | 96 |
| Specificity | 100 | 98 |
| Positive Predictive | 100 | 99 |
| Value | | |
| Negative Predictive | 94 | 95 |
| Value | | |
| miRNA included | 151a-5p, 4454, | 4454, 10b-5p, |
| | 10b-5p, 199a-3p | 199a-5p, 29b-3p |

Data also were analyzed to determine the classification efficiency between: (a) PLS healthy control samples; (b) PLS and ALS; and (c) PLS and Parkinson's Disease. The results are shown in Table 10 below.

TABLE 10

| | | | | | | False | False | | |
|---|---|---|---|---|---|---|---|---|---|
| | | | | | | Negative = | positive = | | |
| PLS | | | | | | have PLS | do not have | | |
| Sample | | | | | | but test says | PLS but test | % overall | |
| Size | Condition | Sensitivity | Specificity | PPV | NPV | not | says you do | accuracy | Kappa |
| 67 | Control, n = 183 | 100 | 100 | 100 | 100 | 0/13 | 0/36 | 100 | 1 |
| 67 | ALS, n = 137 | 92 | 89 | 80 | 96 | 1/13 | 3/27 | 90 | 0.78 |
| 67 | PD, n = 26 | 100 | 100 | 100 | 100 | 0/13 | 0/5 | 100 | 1 |

### Discussion of Results

ALS and/or PLS patients and the neurologists that diagnose them need an ALS and/or PLS diagnostic biomarker. It has been two centuries since Charles Bell first described the symptoms of ALS, and 150 years since Jean-Martin Charcot named the disease. Despite worldwide awareness of ALS and a plethora of scientific research, patients are still faced with a significant diagnosis delay. The rapid progression of the disease increases the need for a quick and accurate diagnostic test from easily obtained biosamples. Adding a blood-based diagnostic test to confirm suspected ALS and/or PLS would help neurologists and benefit patients. The results presented herein have demonstrated the robust, reproducible, sensitive, and accurate ability of an eight-miRNA fingerprint to diagnose ALS and/or PLS, to diagnose subjects at risk of developing ALS and/or PLS, to test subjects to rule out a future ALS and/or PLS diagnosis, and to differentiate between ALS, PLS, and two other neurological diseases, as well as healthy subjects. This miRNA fingerprint (or biomarker) can be utilized as a simple and rapid secondary measure of disease diagnosis following a neurologist's clinical evaluation. The enhanced diagnostic confidence provided by a biomarker would enable neurologists to diagnosis ALS and/or PLS patients, as well as identify subjects at risk of developing ALS and/or PLS, at an earlier stage. This in turn would benefit patients by providing an opportunity for earlier use of disease modifying ALS and/or PLS drugs or other therapies.

In order to qualify as a new diagnostic biomarker with Food and Drug Administration (FDA) approval, four criteria must be met: 1) a needs assessment; 2) context of use; 3) benefit/risk; 4) evidence to support qualification. U.S. Department of Health and Human Services. Biomarker Qualification: Evidentiary Framework Guidance for Industry and Staff 2018, available on the FDA website. The current data evaluates an eight-miRNA ALS and/or PLS diagnostic fingerprint providing evidence for benefit/risk through classification algorithms with good discriminatory power. The high sensitivity, specificity, positive predictive value, and negative predictive value suggests that the benefits of the miRNA fingerprint (biomarker) in conjunction with clinical evaluation would outweigh any risk, particularly given the rapid rate of ALS and/or PLS disease progression. Further evidence to support qualification includes consistent fold-change calculations and highly different fold-regulation between comparison groups as noted by a MannWhitney analyses.

miRNA represent good candidate biomarker targets due to their regulatory roles in essential cell functions. Many miRNA have been identified as dysregulated in ALS and/or PLS patients with on-going research investigating their biomarker potential, as mentioned in the following documents:
Cheng J, Ho W K, et al., “miRNA profiling as a complementary diagnostic tool for amyotrophic lateral sclerosis,” Sci Rep, 13 (1): 13805 (2023);
Cheng Y F, et al., “Signature of miRNAs derived from the circulating exosomes of patients with amyotrophic lateral sclerosis,” Front Aging Neurosci, 15:1106497 (2023);
Gomes B C, et al., “Differential Expression of miRNAs in Amyotrophic Lateral Sclerosis Patients,” Mol Neurobiol., 60 (12): 7104-7117 (2023);
Hur J, Paez-Colasante X, et al., “miRNA analysis reveals novel dysregulated pathways in amyotrophic lateral sclerosis,” Hum Mol Genet., 32 (6): 934-47 (2023);
Koike Y, Onodera O., “Implications of miRNAs dysregulation in amyotrophic lateral sclerosis: Challenging for clinical applications,” Front Neurosci., 17:1131758 (2023).
Liu H, et al., “Systematic review and meta-analysis on microRNAs in amyotrophic lateral sclerosis,” Brain Res Bull., 194:82-89 (2023);
Rizzuti M, Melzi V, et al., “Insights into the identification of a molecular signature for amyotrophic lateral sclerosis exploiting integrated microRNA profiling of iPSC-derived motor neurons and exosomes,” Cell Mol Life Sci., 79 (3): 189 (2022); and
Gama H C, et al., “Systematic review and meta-analysis of dysregulated microRNAs derived from liquid biopsies as biomarkers for amyotrophic lateral sclerosis,” Noncoding RNA Res., Feb. 6 (2024).

Disparity between studies is a concern in miRNA research and can be attributed largely to tissues, methods, sample size, and research focus with a resulting multiplicity of fine detail that can alter the end results between studies. See, e.g., Koshiol J, et al., “Strengths and limitations of laboratory procedures for microRNA detection,” Cancer Epidemiol Biomarkers Prev., 19 (4): 907-911 (2010); and Shademan B, et al., “MicroRNAs as Targets for Cancer Diagnosis: Interests and Limitations,” Adv Pharm Bull., 13 (3): 435 (2023). Reproducibility in miRNA studies can be impacted by: a) pre-analytical variables including sample collection and processing procedures; b) RNA isolation, quantification, and handling methods; c) lack of internal quality control measures; d) data acquisition, processing, and statistical analysis variability. Lakkisto P, et al., “Development of circulating microRNA-based biomarkers for medical decision-making: a friendly reminder of what should NOT be done,” Crit Rev Clin Lab Sci., 60 (2): 141-152 (2023). Furthermore, few studies have been adequately validated using large samples sizes of unique patient cohorts. The nature of ALS and/or PLS as a rare disease further complicates the ability of researchers to find access to large sample sizes which are necessary for the development of robust classification predictions.

The embodiments disclosed herein provide a method to enhance reproducible miRNA discovery intended for clinical applications. This robust ALS and/or PLS miRNA fingerprint (“biomarker”) was identified through a series of steps, and then surprisingly discovering that the biomarker can be identified in a simple blood draw and provide a more accurate diagnosis of ALS and/or PLS than use of neural-enriched extracellular vesicles. The current cohort of samples represent blood plasma collected from several sources using generally accepted plasma collection protocols but without standardization. These samples are thus more heterogeneous in nature, varied in storage duration, ALS and/or PLS stage, and diagnosing neurologist. This diversity in pre-analytical sample sources suggests that the miRNA fingerprint (biomarker) was not sensitive to specific blood plasma collection protocols, an assertion that is consistent with the inventors' prior studies, and increases the likelihood of future reproducibility.

In comparison to all miRNA found to be dysregulated in ALS and/or PLS, the eight-miRNA fingerprint identified herein that has been found to be valuable in various combinations, are a small subset of the those identified. However, the eight-miRNA fingerprint has important links to ALS biological pathways, as shown in Table 11 below. The generic miR-146a, without referring to the specific strand 146a-5p identified, has been confirmed with similar up-regulation in muscle biopsies, brain, and spinal cord, but down regulated in serum. miR-10b-5p did not significantly differ between ALS and controls in muscle biopsies and was found increased in brain, and spinal cord, both differing from the inventors' previous findings in neural-enriched extracellular vesicles from blood plasma. The inventors found miR-29b-3b consistently down-regulated in ALS plasma, but miR-29b was found to be upregulated in ALS muscle biopsy tissues, as disclosed by Russell A P, et al., “Disruption of skeletal muscle mitochondrial network genes and miRNAs in amyotrophic lateral sclerosis,” Neurobiol Dis., 49:107-117 (2013). miR-4454 has been found both in the present examples, and by others (see Saucier D, et al., “Identification of a circulating miRNA signature in extracellular vesicles collected from amyotrophic lateral sclerosis patients,” Brain Res., 1708:100-108 (2019)) to be down-regulated in ALS blood plasma. In serum, however Lo, et al., “Extracellular vesicles in serum and central nervous system tissues contain microRNA signatures in sporadic amyotrophic lateral sclerosis,” Front Mol Neurosci., 14:739016 (2021) found miR-4454 to be up-regulated. miR-151a-5p was shown by others to be up-regulated in early ALS but down-regulated with disease progression. See, e.g., Dobrowolny G, et al., “A longitudinal study defined circulating microRNAs as reliable biomarkers for disease prognosis and progression in ALS human patients,” Cell Death Discov., 7:4 (2021); and Raheja R, et al., “Correlating serum micrornas and clinical parameters in amyotrophic lateral sclerosis,” Muscle Nerve., 58 (2): 261-269 (2018). miR-199a-3p was up-regulated in sporadic-ALS blood plasma, which is consistent with the results herein. Both miR-199a-3p and miR-199a-5p were shown to correlate with clinical ALS parameters. Different tissue types and methods of miRNA detection largely explain the variation noted here between research studies but other factors, as noted above, cannot be ruled out.

The precise function of each miRNA and target pathways related to disease onset and progression are not yet clear. Prior research suggests that these eight-miRNAs affect biological process consistent with neurodegenerative disease affecting such biological processes as oxidative stress, cell viability, motor neuron loss, synaptic transmission, neuron regeneration, neural inflammation, and more (see Table 10 below). However, many miRNA have multiple targets; including several miRNAs from different pathways can increase the robust nature and potential specificity of the test.

TABLE 11
Connection between miRNA biomarkers with ALS biological processes and disease state.

| miRNA (current data + | | |
|---|---|---|
| Banack et al.^{22, 23}) | Biological process/pathways | Link to ALS and Neurodegeneration |
| miR-151a-3p | neuroprotective (Guo et al. 2021) | down-regulated in autism and schizophrenia |
| up-regulated | | (Mundalil Vasu et al. 2014; Moreau et al. 2011) |
| | | dysregulated in PD (Dos Santos et al. 2018) |
| miR-151a-5p | oxidative stress (Pallares-Albanell, 2019) | up-regulated in early-stage ALS but down-regulated |
| up-regulated | cell viability (Pallares-Albanell et al. 2019) | in end-stage ALS (Dobrowolny et al. 2021; Raheja et |
| | | al. 2018) |
| | | down-regulated in PD (He et al. 2021) |
| miR-146a-5p | triggers motor neuron loss (Sison et al. 2017) | sALS: pathogenic, found in white blood cells, CSF, |
| up-regulated | regulates neurofilament protein (Campos-Melo et | and spinal cord (Butovsky et al. 2012) |
| | al. 2013) | up-regulated in ALS muscle biopsy (Pegoraro et al. |
| | increases synaptic transmission (Chen et al. 2013) | 2017) |
| | reduces synaptic plasticity (Benoist et al. 2013) | up regulated in ALS brain and spinal cord (Alvia et |
| | contributes to neuroinflammation (Varma-Doyle | al. 2022) |
| | et al. 2021; Etzrodt et al. 2012; Cui et al. 2010) | down-regulated in ALS serum (Tasca et al. 2016) |
| | may function to alleviate neuropathic pain (Lu, | down-regulated in AD plasma or serum (Cui et al. |
| | Cao et al. 2015) | 2010; Cogswell et al. 2008; Sethi and Lukiw 2009; |
| | pre-miR-146a regulates mitochondria and | Kiko et al. 2014; Müller et al. 2014; Dong et al. 2015) |
| | inflammation (Barbosa et al. 2021) | up-regulated in AD brain, CSF (Alexandrov et al. |
| | impacts cellular bioenergetics (Kim et al. 2022) | 2012; Lukiw et al. 2012; Denk et al. 2015; Kim et al. |
| | | 2021;) |
| | | up-regulated in multiple sclerosis CSF (Muñoz-San |
| | | Martín 2019) |
| miR4454 | predicted to impact neurogenesis, synapse | up-regulated in ALS serum (Lo et al. 2021) |
| down-regulated | formation, and motor neuron integrity (Lo et al. | specific to ALS, not dysregulated in PD (Chen et al. |
| | 2021) | 2021) |
| | | found down-regulated in ALS blood plasma |
| | | (Saucier et al. 2019) |
| miR-10b-5p | increases BDNF (Varendi et al. 2014) | increased BDNF in ALS lymphocytes (Li et al. |
| down-regulated | positive effect on memory and learning (Li et al. | 2012; Buchman et al. 2016; Sadanand et al. 2018) |
| | 2012; Buchman et al. 2016; Sadanand et al. 2018) | up regulated in ALS brain and spinal cord (Alvia et |
| | positive effect on synaptogenesis and neuron | al. 2022) |
| | survival (Li et al. 2012; Buchman et al. 2016; | dysregulated in PD (Dos Santos et al. 2018) |
| | Sadanand et al. 2018) | up-regulated in HD (Hoss et al. 2015) |
| | down-regulated in myoblasts proliferation and | |
| | up-regulated in myoblasts differentiation (Ge et al. | |
| | 2019) | |
| | down-regulation enhances HOXD10 | |
| | and inactivates Rho/ROCK signaling pathway | |
| | resulting in reduced neuronal apoptosis, | |
| | inflammation, and oxidative stress (Ruan et al. | |
| | 2021) | |
| miR-199a-3p | negative effect on regeneration of damaged | correlated with clinical ALS parameters (Raheja et |
| up-regulated | neurons (Liu et al. 2012) | al. 2018) |
| | decreases mTOR negatively affecting axon | up-regulated in sALS plasma (Cheng et al. 2023) |
| | regeneration and plasticity (Liu et al. 2012, Kar et | down-regulated in PD (Hoss et al. 2015, Zhou et al. |
| | al. 2021) | 2021) |
| miR-199a-5p | protective in spinal cord injury models (Bao et al. | correlated with clinical ALS parameters (Raheja et |
| up-regulated | 2018, Zhong et al. 2020, Li et al. 2019) | al. 2018) |
| | promotes neurogenesis and neuronal | expression differentiates AD (Raheja et al. 2018) |
| | differentiation (Ji et al. 2023) | |
| miR-29b-3p | regulates pro-apoptotic/anti-apoptotic pathways | up-regulated in ALS muscle biopsy (Russell et al. |
| down-regulated | (Slusarz et al. 2015) | 2013) |

Abbreviations: ALS = Amyotrophic Lateral Sclerosis, sALS = sporadic ALS, AD = Alzheimer's Disease, BDNF = brain-derived neurotrophic factor; CSF = cerebrospinal fluid; HOXD10 = homeobox D10; mTOR = mammalian target of rapamycin; PD = Parkinson's Disease; Rho/ROCK = Rho-associated protein kinase.

Of particular research interest is finding blood-based biomarkers that can enhance ALS and/or PLS clinical trials. Seven blood protein biomarkers are currently being used in on-going ALS trials but only one, high-sensitivity cardiac troponin T (hs-cTnT), has promising potential as a diagnostic. See Kläppe U, et al., “Cardiac troponin T is elevated and increases longitudinally in ALS patients,” Amyotroph Lateral Scler Frontotemporal Degener., 23 (1-2): 58-65 (2022). It has not been shown to be as accurate as the current miRNA fingerprint (biomarker) with the ability to distinguish ALS from ALS mimics noted as 0.70 AUC (95% CI=0.61-0.79) and its ability to differentiate ALS and/or PLS from healthy controls as 0.88 AUC (95% CI=0.70-0.97). The majority of research to date on ALS diagnostics has been on neurofilament light chain which was initially considered as a diagnostic. However, since neurofilament light chain concentrations have been found to increase in blood plasma associated with many neurodegenerative diseases, it is perhaps better suited as a surrogate end-point in clinical trials. This is the current context in which neurofilaments are being employed.

In contrast to known diagnostic methods, the present inventors have discovered the ability of this eight-miRNA fingerprint (biomarker) for ALS and/or PLS diagnosis with high sensitivity and accuracy. The inventors discovered highly significant differences in the miRNA expression between ALS and/or PLS, Parkinson's disease and primary lateral sclerosis, as shown above.

The eight-miRNA fingerprint described herein provides evidence of a strong relationship between this diagnostic biomarker and the gold standard method of clinical disease diagnosis. The analytical performance for NEE is good in three separate classification analyses (Table 6, Sensitivity=82, 96, 100; Specificity=97, 97, 97%) with important predictive value (PPV=95-96%; NPV=87-100%), while the analytical performance for blood-based detection without exosome isolation is superior in at least two different classification analysis (Table 9, Sensitivity=95, 96; Specificity=100, 98%) with important predictive value (PPV=98-100%; NPV=94-95%). The use of independent cohort groups in the machine learning algorithm, with one cohort used for training and another cohort for testing, provides support for the predictive value of the test. The significant differences found in the fold-regulation between ALS, Parkinson's disease, and primary lateral sclerosis, further support the utility of this biomarker. Four independent experiments have tested the eight-miRNA fingerprint to compare ALS and non-neurological controls samples which used different patient samples, for a total sample size of 471. The analytical validation of the miRNA fingerprint in this study and prior research establishes that the biomarker test is both accurate and reproducible on blood plasma collected using multiple collection protocols. It is not sensitive to variation in standard blood plasma collection, storage, or processing conditions which increases its potential for clinical diagnostic evaluation. Further validation of this biomarker using samples from prospective and longitudinal studies, as well additional neurological controls and ALS-mimic diseases, would aid its future use and accelerate its introduction into the clinic and clinical trials.

Table 12 below provides the miRNA sequences of the miRNA fingerprint

TABLE 12
miRNA Sequences

| | | Mirabase | |
|---|---|---|---|
| mi-RNA | SEQ | (www.mirabase.org) | |
| name | ID NO: | Accession No. | Sequence |
| miR-146a-5p | 1 | MI0000477 | TGAGAACTGAATTCCATGGGTT |
| miR-199a-3p | 2 | MI0000242 | ACAGTAGTCTGCACATTGGTTA |
| miR-4454 | 3 | MI0016800 | GGATCCGAGTCACGGCACCA |
| miR-10b-5p | 4 | MI0000267 | TACCCTGTAGAACCGAATTTGTG |
| miR-29b-3p | 5 | MI0000105 | TAGCACCATTTGAAATCAGTGTT |
| miR-151a-3p | 6 | MI0000809 | CTAGACTGAAGCTCCTTGAGG |
| miR-151a-5p | 7 | MI0000809 | TCGAGGAGCTCACAGTCTAGT |
| miR-199a-5p | 8 | MI0000809 | CCCAGTGTTCAGACTACCTGTTC |

**Any or all “T” nucleotide bases in the sequences of the miRNAs shown in Table 8 above may be substituted with a “U” nucleotide base.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The embodiments are generally disclosed herein using affirmative language to describe the numerous features and aspects thereof. The embodiments also specifically include features in which particular subject matter is excluded, in full or in part, such as substances or materials, method steps and conditions, protocols, or procedures. For example, in certain embodiments or aspects of the invention, materials and/or method steps are excluded. Thus, even though the embodiments are generally not expressed herein in terms of what they do not include, aspects that are not expressly excluded are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. A composition “consisting essentially of” refers to a composition that includes only the active ingredients claimed (e.g., active ingredient (AI) or active pharmaceutical ingredient (API); e.g., L-serine, a salt, metabolic precursor, derivative or conjugate thereof); which composition may include other ingredients such as formulation materials, excipients, additives, carriers, preservatives, diluents, solvents, fillers, salts, buffers, coatings, binders, and lubricating agents; and which composition excludes other APIs not claimed.

The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

What is claimed is:

1. A method of identifying a subject who has, or is at risk of developing Amyotrophic Lateral Sclerosis (ALS) comprising:
(a) determining a presence or amount of two or more micro-RNAs (miRNAs) in a subject's circulating blood, wherein the two or more miRNA are selected from the group consisting of miR-199a-3p, miR-4454, miR-10b-5p, miR-151a-5p, miR-199a-5p, miR-151a-3p, miR-146a-5p, andr miR-29b-3p, without determining a presence or amount of the miRNAs from neural-derived exosomes; and
(b) determining if the subject has, or is at risk of developing ALS according to the presence or amount of the two or more miRNAs in the sample.

2. The method of claim 1, wherein the method further comprises a method of preventing or treating a motor neuron disease in a subject who has, or is at risk of developing ALS, the method comprising:
(c) administering a therapeutically effective amount of a motor neuron disease drug to the subject when the determining of (b) determines that the subject has, or is at risk of developing ALS.

3. The method of claim 2, wherein the motor neuron disease drug is selected from L-serine, ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenibut, imagabalin, atagabalin, 4-methylpregabalin, PD-217,014, riluzole, edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine, a cholinesterase inhibitor, memantine, an antidepressant, an N-methyl D-aspartate (NMDA) antagonist, an omega-3 fatty acid, curcumin, or a curcumin derivative, vitamin E, a sleep aid, an anti-anxiety drug, an anti-convulsant, an anti-psychotic, carbidopa-levodopa, amantadine, a dopamine agonists, a MAO B inhibitor, a Catechol O-methyltransferase (COMT) inhibitor, and an anticholinerigic.

4. The method of claim 1, wherein the amount of the micro-RNAs determined in (a) is at least 1.1-fold higher or lower than a baseline amount, thereby indicating the subject has, or is a risk of developing, ALS.

5. The method of claim 1, wherein the subject is a human.

6. The method of a claim 1, wherein the subject is asymptomatic for ALS.

7. The method of claim 4, wherein the baseline amount is an average, mean or absolute amount of any one of the miRNAs present in a healthy control subject.

8. The method of claim 1, further comprising determining the absence of ALS in the subject according to the presence or amount of the two or more miRNAs in the sample.

9. The method of claim 1, further comprising monitoring the progression of ALS in the subject, wherein the method is conducted two or more times for the subject.

10. The method of claim 1, wherein the subject is not diagnosed with ALS prior to the determining of (a) or (b).

11. The method of claim 2, wherein the treating of ALS comprises inhibiting or delaying the onset or progression of ALS.

12. A method of preventing or treating Amyotrophic Lateral Sclerosis (ALS) in a subject who has, or is at risk of developing ALS, the method comprising:
(a) determining a presence or amount of two or more micro-RNAs (miRNAs) in a subject's circulating blood, wherein the two or more miRNA are selected from the group consisting of miR-199a-3p, miR-4454, miR-10b-5p, miR-151a-5p, miR-199a-5p, miR-151a-3p, miR-146a-5p, andr miR-29b-3p, without determining a presence or amount of the miRNAs from neural-derived exosomes;
(b) determining if the subject has, or is at risk of developing ALS according to the presence or amount of the two or more miRNAs in the sample; and
(c) administering a therapeutically effective amount of a motor neuron disease drug to the subject when the determining of (b) determines that the subject has, or is at risk of developing ALS.

13. The method of claim 12, wherein the motor neuron disease drug is selected from L-serine, ralitoline, phenytoin, lamotrigine, carbamazepine, lidocaine, tetrodotoxin, nitroindazole, a sulforaphane or sulforaphane analogue, gabapentin, pregabalin, Mirogabalin, gabapentin enacarbil, phenibut, imagabalin, atagabalin, 4-methylpregabalin, PD-217,014, riluzole, edaravone, tetrabenazine, haloperidol, risperidone, quetiapine, amantadine, levetiracetam, clonazepam, citalopram, escitalopram, fluoxetine, sertraline, quetiapine, risperidone, olanzapine, valproate, carbamazepine, lamotrigine, a vaccine, a cholinesterase inhibitor, memantine, an antidepressant, an N-methyl D-aspartate (NMDA) antagonist, an omega-3 fatty acid, curcumin, or a curcumin derivative, vitamin E, a sleep aid, an anti-anxiety drug, an anti-convulsant, an anti-psychotic, carbidopa-levodopa, amantadine, a dopamine agonists, a MAO B inhibitor, a Catechol O-methyltransferase (COMT) inhibitor, and an anticholinerigic.

14. The method of claim 12, wherein the amount of the micro-RNAs determined in (a) is at least 1.1-fold higher or lower than a baseline amount, thereby indicating the subject has, or is a risk of developing, ALS.

15. The method of claim 12, wherein the subject is a human.

16. The method of claim 14, wherein the baseline amount is an average, mean or absolute amount of any one of the miRNAs present in a healthy control subject.

17. The method of claim 12, wherein the treating of ALS comprises inhibiting or delaying the onset or progression of ALS.
